(19) United States
(12) Patent Application Publication
(10) Pub. No.: US 2025/0257045 A1
(43) Pub. Date: Aug. 14, 2025

(54) CAGED GLUTAMATE UNLEASHED BY BLUE AND GREEN LIGHT: INNOVATIVE PHOTOSENSITIVE CONTROL

(71) Applicant: Florida Institute of Technology, Inc., Melbourne, FL (US)

(72) Inventors: Nishal M. Egodawaththa, Melbourne, FL (US); Nasri Nesnas, West Melbourne, FL (US); Jingxuan Ma, Beijing (CN)

(21) Appl. No.: 18/975,174

(22) Filed: Dec. 10, 2024

Related U.S. Application Data
(60) Provisional application No. 63/552,557, filed on Feb. 12, 2024.

(51) Int. Cl. C07D 311/18; C07F 5/02

(52) U.S. Cl. CPC C07D 311/18; C07F 5/022; C07F 5/027

(57) ABSTRACT

This disclosure teaches the development of photosensitive cages for neurotransmitters, specifically Glutamate (Glu). These compounds are designed to be responsive to visible light, particularly blue and green wavelengths, offering a unique approach for controlled neuro-agonist release in biological and experimental applications. The invention also encompasses novel caging strategies, such as the use of the thiocoumarin (TC) system and BODIPY systems, with various substitutions, which enhance the flexibility and efficiency of these caged compounds.

### CROSS REFERENCE TO RELATED APPLICATIONS AND INCORPORATION BY REFERENCE

This non-provisional patent application claims benefit of priority to U.S. provisional patent application No. 63/552,557, entitled CAGED GLUTAMATE UNLEASHED BY BLUE AND GREEN LIGHT: INNOVATIVE PHOTOSENSITIVE CONTROL, which was filed in the United States Patent and Trademark Office (USPTO) on Feb. 12, 2024, which is hereby incorporated by reference herein in its entirety.

### STATEMENT REGARDING FEDERALLY SPONSORED RESEARCH OR DEVELOPMENT

This invention was made with government support under Grant Number 2R15GM112119-02 awarded by the NIH. The government has certain rights in the invention.

### FIELD OF THE INVENTION

The invention described is primarily related to the synthesis of photosensitive cages for neurotransmitters, specifically Glutamate (Glu). These compounds are designed to be responsive to visible light, particularly blue and green wavelengths, offering a unique approach for controlled neuro-agonist release in biological and experimental applications. The invention also encompasses novel caging strategies, such as the use of the thiocoumarin (TC) system and BODIPY (boron dipyridyl) systems, with various substitutions, which enhance the flexibility and efficiency of these caged compounds.

### BACKGROUND

Glutamate (Glu) is an excitatory neurotransmitter that plays a critical role in memory. Brain mapping activities of such pathways relied heavily on the ability to release Glu with spatiotemporal precision. Several photo-protecting groups (PPGs), referred to as photocages or cages, were designed to accomplish the release of Glu upon irradiation. Previously reported Glu cages responded to UV upon irradiation with single photons, which limited their use in vivo experiments due to cytotoxicity. Other caged designs suffered from lower quantum efficiency (QE) of release, thus necessitating higher concentrations and/or longer photoirradiation times. There have been limited examples of cages that respond to visible light with single photon irradiation.

Notably, several cages for Glu have been crafted to exhibit superior photophysical properties. Among these, CDNI-Glu stands out as one of the most efficient caged Glu compounds, boasting a notable quantum yield (Φ) of 0.5. While it primarily absorbs light at 350 nm, remarkable advancements have allowed it to be responsive to both 2-photon and, more recently, related designs responded to 3-photon irradiation, paving the way for far-infrared control. This development holds great promise for biological applications by minimizing phototoxicity and harnessing the enhanced tissue-penetrating potential of longer wavelengths.

It is essential that caged designs have properties that render them useful in neuroscience applications. This includes absorption wavelengths safe for biological tissues, preferably above 350 nm. The absorption efficiency and responsiveness can be reflected by measurement of the molar attenuation constant (ε) and the quantum yield (Φ). The cross product of the two is a parameter known as the photo-cross-section, which reflects the efficiency of light responsiveness. The quantum yield can be computed through kinetics experiments that measure the rate of uncaging. Ideal cages should also have good water solubility as well as minimal interference of interactions with other receptors.

Glutamate (Glu) is the most common excitatory neurotransmitter that plays an important role in memory. It is known in the prior art that cages for Glu have been successfully designed with superior photophysical properties. Among the most efficient caged Glu is CDNI-Glu, with a quantum yield of 0.5. Although the cage has an absorption maximum at 350 nm, it has been shown to respond to 2-photon irradiation.

However, there are no examples of organic caged Glu compounds that respond to single photons in visible wavelengths/light spectrum; (the only previous example reported is based on the precious metal Ruthenium with a reported quantum yield of 0.13). Therefore, what is needed in the art is caged Glutamate (Glu) compounds responsive to visible light. The ability to respond to visible light opens up various possibilities for controlled neuro-agonist release using different wavelengths. The invention of caged Glutamate (Glu) compounds responsive to visible light brings about significant advantages in the field of neurobiology and optogenetics. These advantages include the ability to trigger caged Glu with visible light, specifically blue and green wavelengths, offering precise control and versatility in neuro-agonist release. This not only reduces phototoxicity but also enables tailored neuro-agonist release, opening up new possibilities for controlled experimentation and commercial applications.

### SUMMARY OF THE INVENTION

The present disclosure teaches the preparation of 11 distinct caged Glu molecules, each capable of being uncaged using visible light. One of these cage designs is founded on the thiocoumarin (TC) system and responds to blue light at 467 nm, while the remaining ten caged Glu variants are based on the BODIPY (boron dipyridyl) system and sensitized to green light across a range of wavelengths from 520 to 560 nm.

An object of the present invention is directed to the synthesis of 11 caged Glu examples that respond to two visible wavelengths, 467 nm (thiocoumarin based) and 520-560 nm (BODIPY based). The Me-BODIPY-Br-Glu cage shows the most efficient Glu release with a QE of 0.65. Similar caged designs can be extended to the inhibitory neurotransmitter, GABA. This would enable the use of two visible wavelengths to modulate the release of excitatory and inhibitory neurotransmitters upon demand via optical control.

Referring to FIG. 1, an object of the present invention is to provide novel caged Glutamate molecules 1-11. These 11 variants responded to visible light with wavelengths ranging from 470-560 nm and with quantum yields that range from 0.0001 to 0.65.

The present invention discloses 11 caged Glutamate (Glu) molecules that respond to visible light, which is a significant advancement in the art. The ability to respond to visible light opens up various possibilities for controlled neuro-agonist release using different wavelengths. The invention of caged Glutamate (Glu) compounds responsive to visible light brings about significant advantages in the field of neurobiology and optogenetics. These advantages include the ability to trigger caged Glu with visible light, specifically blue and green wavelengths, offering precise control and versatility in neuro-agonist release. This not only reduces phototoxicity but also enables tailored neuro-agonist release.

The invention disclosure utilizes two main caging approaches, one based on the thiocoumarin (TC) system and the other on BODIPY systems. The availability of two distinct caging systems, one based on the thiocoumarin (TC) system and the other on BODIPY systems, enhances experimental flexibility, providing a broad range of options for researchers and commercial applications. Systematic comparisons of caged systems help researchers select the most efficient compounds for their specific needs, streamlining decision-making and promoting informed choices in experimental design. Additionally, the incorporation of heavy atom substitutions in some caged Glu compounds, leading to a redshift in absorption wavelength, enhances the utility of these compounds for specific applications, contributing to more precise control in experiments. This diversity in caging strategies provides a range of options for commercial applications and researchers.

The blue cage is based on the thiocoumarin (TC) system whereas the green light responsive cage is based on the BODIPY (boron dipyridyl) system. Referring to FIGS. 1 and 9, BODIPY has two positions that can be substituted during their assembly, and they include the third position on the pyrrole ring, which becomes the C-2 and C-6 positions on the BODIPY, and the substituent at the boron site (originally an F which can be substituted with a CH₃ group). The abbreviated name will reflect these variations with the boron substituent in the prefix of the BODIPY name and the C-2/C-6 substituent in the suffix of the name, e.g., Me-BODIPY-Br-Glu for compound 8. Cages attached to the amine functional group of Glu will be distinguished with addition of “N” preceding Glu, e.g., Me-BODIPY-Br-N-Glu for 3.

The disclosure compares the performance of caged Glu molecules using different caging strategies, such as thiocoumarin and BODIPY, with various substitutions, including halogen atoms and methyl groups. This comparative analysis helps commercial entities and researchers select the most suitable caged compound for their specific needs.

The introduction of heavy atom substitutions in the BODIPY caged Glu compounds is noteworthy. The addition of bromine or iodine atoms leads to a significant redshift (bathochromic shift) in the absorption wavelength, enhancing the utility of these compounds for specific applications. The incorporation of heavy atom substitutions, such as bromine or iodine, to achieve a bathochromic shift and improve quantum yields is a unique and innovative approach. These heavy atom-modified caged Glu compounds have not been previously reported.

This disclosure teaches that replacing fluorine atoms on the boron of classic BODIPY with methyl groups significantly improves the quantum yields (QY) of caged Glu molecules. This modification enhances the efficiency of uncaging and makes these caged compounds more practical for various applications.

The introduction of methylated BODIPY compounds with improved quantum yields (QY) significantly improves the efficiency of neuro-agonist release, making them more practical for biological applications. This modification addresses the limitations of relatively low QY in some caged Glu systems, ultimately enhancing their effectiveness.

The caged Glu compounds described herein respond to visible light, whereas many existing caged compounds primarily require UV or near-UV light for uncaging. The use of visible light for uncaging allows for more precise and controlled activation of Glu in biological systems, reducing off-target effects and minimizing phototoxicity. This novel feature expands the range of wavelengths that can be used for controlled release of Glu.

The disclosure of Glu caging strategies (thiocoumarin and BODIPY) with various substitutions and their detailed comparison is novel. This diversity provides commercial entities and researchers with a broader toolkit.

The incorporation of heavy atom substitutions, such as bromine or iodine, to achieve a bathochromic shift and improve quantum yields is a novel and innovative approach. The incorporation of heavy atoms into the caged Glu compounds not only leads to a redshift but also significantly improves quantum yields, making them highly efficient for research and practical commercial applications. These heavy atom-modified caged Glu compounds have not been previously reported.

This disclosure highlights the advantages of using methylated BODIPY for caging Glu, which significantly improves quantum yields. This innovation can enhance the efficiency of neuro-agonist release in research and practical commercial applications.

Glu has three functional groups: an a-amino, an a-carboxylic acid, and a y-carboxylic acid group. Protecting any of the three groups leads to the silencing of neuro agonist behavior. In our previous work with CDNI cages, we noted that the efficiency of uncaging is dramatically lower when the amino group is caged. However, with the 11 caged Glu reported here, the functionalization of the amino group did not negatively impact the QE of uncaging. This is likely due to the cage being attached via an oxygen atom (carbamate bond). Illustrated in FIG. 1 are the caged Glu compounds 1-11. The corresponding synthetic yields of the caging step in the synthesis of compounds 1-11 are: Compound 1—38%; Compound 2—52%; Compound 3—36%; Compound 4—37%; Compound 5—21%; Compound 6—33%; Compound 7—20%; Compound 8—22%; Compound 9—27%; Compound 10—25%; Compound 11—29%.

The coumarin scaffold has demonstrated its potential as a cage that absorbs in the blue light. Notably, it has been observed that substituting the lactone oxygen with sulfur, creating thiocoumarin, results in a significant bathochromic shift, shifting it towards longer wavelengths (redshift) by approximately 20 nm. In our research, we successfully synthesized an amino-caged Glu employing the thiocoumarin system, resulting in the previously unreported compound N-TC-Glu, shown as compound 1 in FIGS. 1 & 2.

Still referring to FIG. 2, activation of the amino group is achieved via a reaction of the bis-BOC protected Glu 12 with triphosgene leading to the isocyanate 13 which can be reacted subsequently with the thiocoumarin primary alcohol 18. 7-Diethylamino-4-methylcoumarin, 14, can be smoothly oxidized to the aldehyde using SeO₂, followed by reduction with NaBH₄ to generate the 4-hydroxymethylated 15 in 42% overall yield. In order to convert the coumarin system to the thiocoumarin system, it was necessary to first protect the primary alcohol of hydroxymethyl 15 with acetate using classic EDC coupling chemistry with acetic acid. Sulfurization of the lactone was achieved with 69% yield using Lawesson's reagent. Deprotection of the acetate was accomplished via acid hydrolysis of the acetate prior to reaction with isocyanate 13, to afford 19. Deprotection of the bis-BOC was achieved in 50% yield to generate N-TC-Glu, 1.

The synthesis of these caged compounds may be intricate. Some caged compounds depend on specific substitutions (e.g., heavy atoms) to achieve desired properties, limiting their versatility. While modifications improve QY in some compounds, others may still have relatively low QY, which can affect their efficiency. The response of caged compounds to light can vary, necessitating careful selection based on any specific requirements.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating exemplary embodiments of the invention and are not to be construed as limiting the invention. In the drawings:

FIG. 1 depicts the 11 designed caged GLU molecules of the present invention. N-caged Glu (blue light responsive 1 and green light responsive 2-5) and y-carboxy caged Glu (green light responsive 6-11).

FIG. 2 depicts the scheme for synthesis of an amino-caged Glu using the thiocoumarin system forming the unreported compound N-TC-Glu, shown as compound 1.

FIG. 3 depicts the scheme for synthesis of hydroxymethyl-BODIPY compounds.

FIG. 4 depicts the scheme for synthesis of BODIPY-N-Glu caged molecules.

FIG. 5 depicts the scheme for synthesis of BODIPY-Glu caged molecules.

FIG. 6 depicts plots showing a UV comparison of a. TC-N-Glu b. BODIPY-N-Glu c. BODIPY-Glu.

FIG. 7 depicts the 1H NMR analysis of progressive photolysis of the caged molecules: TC-N-Glu (1) was irradiated at 467 nm for 60 s intervals.

FIG. 8 depicts the 1H NMR analysis of progressive photolysis of the caged molecules: BODIPY-(N)-Glu (6) was irradiated at 520 nm for 120 s intervals.

FIG. 9 depicts blue and green light responsive caged Glu molecules.

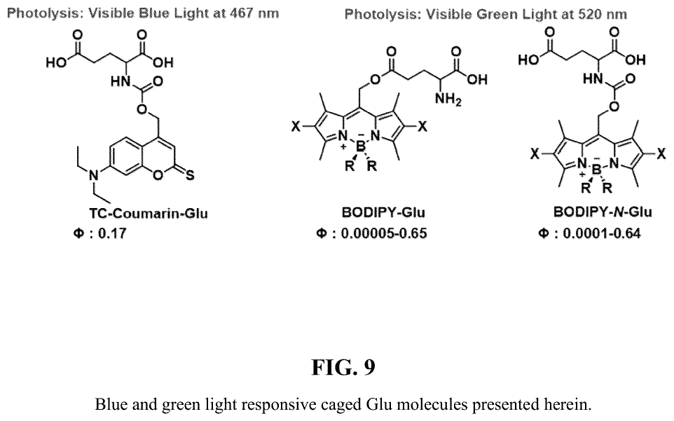

FIG. 10 depicts irradiation rigs used in photochemical studies of Caged Glutamate compounds 1-11.

FIG. 11 depicts results of the UV spectra for progressive photolysis of Caged compound 1.

FIG. 12 depicts results of the UV spectra for progressive photolysis of Caged compound 2.

FIG. 13 depicts results of the UV spectra for progressive photolysis of Caged compound 3.

FIG. 14 depicts results of the UV spectra for progressive photolysis of Caged compound 4.

FIG. 15 depicts results of the UV spectra for progressive photolysis of Caged compound 5.

FIG. 16 depicts results of the UV spectra for progressive photolysis of Caged compound 6.

FIG. 17 depicts results of the UV spectra for progressive photolysis of Caged compound 7.

FIG. 18 depicts results of the UV spectra for progressive photolysis of Caged compound 8.

FIG. 19 depicts results of the UV spectra for progressive photolysis of Caged compound 9.

FIG. 20 depicts results of the UV spectra for progressive photolysis of Caged compound 10.

FIG. 21 depicts results of the UV spectra for progressive photolysis of Caged compound 11.

In the Figures of the drawings, like callouts refer to like elements.

### DETAILED DESCRIPTION OF THE INVENTION

The following documentation provides a detailed description of the invention.

Although a detailed description as provided in this application contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention. Thus, the scope of the invention should be determined by the appended claims and their legal equivalents, and not merely by the preferred examples or embodiments given.

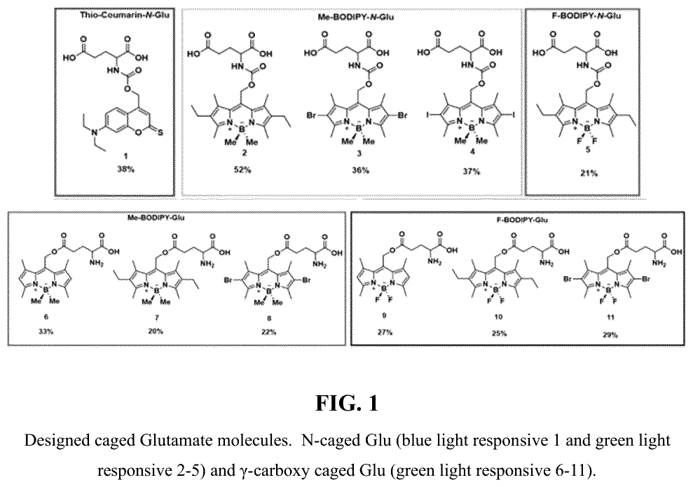

This disclosure is directed to novel cage systems for Glu, enabling uncaging with blue and green light. Referring to FIG. 1, this disclosure teaches the preparation of 11 distinct caged Glu molecules 1-11, each capable of being uncaged using visible light. One of these cage designs is founded on thiocoumarin (TC) and responds to blue light at 467 nm, while the remaining ten caged Glu variants are sensitized to green light across a range of wavelengths from 520 to 560 nm.

### Materials and Methods

All starting materials were purchased from Fisher, Acros, TCI, or Sigma-Aldrich in highly purified form, and used with no further purification, unless otherwise stated. The solvents used are dichloromethane (CH₂Cl₂), hexane (C₆H₁₄), acetone (C₃H₆₀), methanol (MeOH), and ethyl acetate (EtOAc). Anhydrous toluene was obtained using dry molecular sieves, while dry CH₂Cl₂ and ether were directly obtained from the solvent purification system (MBRAUN, MB-SPS) via activated alumina column. The reactions were performed under N₂ or Ar using the standard Schlenk line technique. Reaction vessels were either flame-dried or oven-dried followed by three repeating cycles of alternating vacuum and N₂/Ar flushes. Monitoring reaction progress was conducted using thin-layer chromatography plates (TLCs) (EMD TLC Silica 60 F254) and observed under short or long wavelength UV light. Purification for intermediates was achieved by column chromatography using 60 Å silica gel (Fisher Scientific). The final product of each caged molecule was purified using reversed-phase HPLC performed on Waters 2695 Separation Module and the Waters 996 Photodiode Array Detector. The HPLC column was XSELECT C18 OBD preparative column (10×250 mm) and the mobile phase was either HPLC-grade acetonitrile (ACN) or methanol (MeOH), and Millipore water with trifluoroacetic acid (TFA) (0.1%).

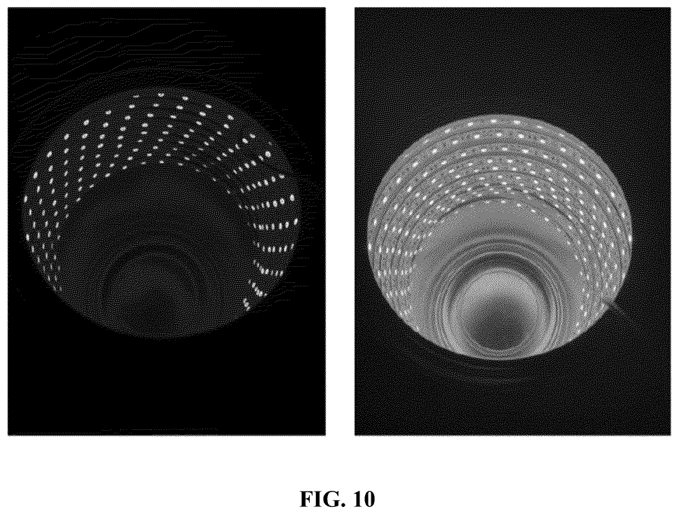

After purification, the organic solvent was removed by rotovap under vacuum and the remaining aqueous concentrate was subjected to a freeze-drying system (lyophilizer-Freezone 4.5 made by LABCONCO) to isolate the purified final product. The preparation of the caged compounds and their corresponding purifications were conducted under red light in the dark. Final products were covered with aluminum foil and stored at −20° C. until further needed. Each product was characterized by Proton Nuclear Magnetic Resonance (hereinafter “1H NMR”) and Carbon-13 Nuclear Magnetic Resonance (hereinafter “¹³C NMR”) using Bruker 400 MHZ NMR spectrometer instrumentation. NMR solvents such as CDCl₃ and (CD₃)₂CO were purchased from the Cambridge Isotope Laboratories. Chemical shifts were reported relative to the internal standard peak of tetramethylsilane (TMS). UV-Vis spectra were obtained using the Agilent 8453 diode array spectrophotometer. High-resolution mass spectrometry (HRMS) was measured using JEOL DART-AccuTOF mass spectrometer, whereas low-resolution mass spectrometry was obtained using Agilent LC-MS measurements ESI+ and 6120 quadrupoles. Referring to FIG. 10, irradiation rigs constructed of two round metal containers stacked with blue LED strips (467 nm) and green LED strips (520 nm) on their inner walls were used as photoreactors to irradiate the caged molecules in photochemical studies. The light flux was determined using liquid-phase potassium ferrioxalate actinomentry.

### Synthesis of TC-N-Glu Cage 1

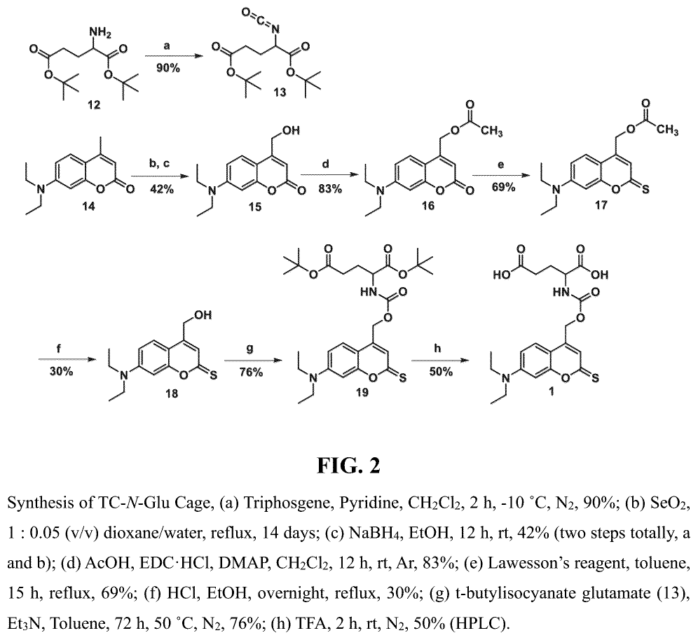

Referring to FIG. 2, shown is a procedure and intermediates for synthesizing TC-N-Glu 1. Activation of the amino group is achieved via a reaction of the bis-BOC protected Glu 12 with triphosgene leading to the isocyanate 13 which can be reacted subsequently with the thiocoumarin primary alcohol 18. 7-Diethylamino-4-methylcoumarin 14 can be smoothly oxidized to the aldehyde using SeO₂, followed by reduction with NaBH₄ to generate the 4-hydroxymethylated 15 in 42% overall yield. In order to convert the coumarin system to the thiocoumarin system, it was necessary to first protect the primary alcohol of hydroxymethyl 15 with acetate using classic EDC coupling chemistry with acetic acid. Sulfurization of the lactone was achieved with 69% yield using Lawesson's reagent. Deprotection of the acetate was accomplished via acid hydrolysis of the acetate prior to reaction with isocyanate 13, to afford 19. Deprotection of the bis-BOC was achieved in 50% yield to generate N-TC-Glu, 1.

Synthesis of tert-Butyl-isocyanate-glutamate 13 was accomplished by the following steps. tert-Butyl-glutamic acid (2.00 g, 6.76 mmol) and pyridine (2.2 mL, 27.31 mmol) were added in the flame-dried flask with CH₂Cl₂ (200 mL), cooled at −10° C. under N₂ for 30 min. Then triphosgene (1.35 g, 4.54 mmol) was added by syringe over 30 seconds, and stirred for 2 hours at −10° C. After the completion of the reaction time, the crude product was washed twice with cold 0.1 M HCl followed by saturated NaCl solution with crushed ice. The organic layer was dried over Na₂SO4. Then the residue was concentrated by vacuum and the product used immediately in the following steps.

Synthesis of 7-(N, N-Diethylamino)-4-hydroxymethylcoumarin 15 was accomplished by the following steps. 7-diethylamino-4-methylcoumarin 14 (5.0 g, 22 mmol) was dissolved in dioxane and water (30:1 (v/v)), followed by selenium dioxide (7.0 g, 64 mmol). The mixture was stirred and refluxed for 14 days. The reaction progress was monitored by TLC and after complete consumption of the starting material, the suspension was cooled to room temperature and then filtered and concentrated under vacuum. The residue was then dissolved in absolute ethanol, placed in a flame-dried flask, and NaBH₄ (832.3 mg, 22 mmol) was added. The solution was stirred for 12 hours at room temperature. Upon completion, the reaction was quenched with 1 M HCl and diluted with water. After CH₂Cl₂ extraction, the organic layer was washed with water and dried with anhydrous Na₂SO4. Finally, the solvent was removed under vacuum, and the purified yellow solid was obtained by column chromatography (CH₂Cl₂: acetone=4:1, 2.98 g, 11.9 mmol, yield %=42%). Melting point (mp), 142-145° C. The characterization using NMR Spectroscopy and Mass Spectrometry yielded the following: 1H NMR (400 MHZ, acetone-d): δ 7.30 (d, J=9.0 Hz, 1H), 6.54 (dd, J=9.0, 2.5 Hz, 1H), 6.44 (t, J=2.1 Hz, 1H), 6.28 (s, 1H), 4.82 (d, J=1.5 Hz, 2H), 3.38 (q, J=7.1 Hz, 4H), 1.18 (t, J=7.1 Hz, 6H). LCMS (m/z) calculated for C₁₄H₁₇NO₃⁺ 248.1 found 248.1.

Synthesis of 7-Diethylamino-4-methylacetoate-coumarin 16 was accomplished by the following steps. 7-(N, N-Diethylamino)-4-hydroxymethylcoumarin 15 (1 g, 4.0 mmol) was added to a flame-dried flask and dissolved in anhydrous CH₂Cl₂ (200 mL). Then acetic acid (4.8 mmol, 0.28 mL) and 4-dimethylaminopyridine (DMAP, 0.6 g, 4.8 mmol) was added and stirred under N₂. The ice bath was employed to cool down the solution to 0° C. Thereafter, 1-(3-dimethylaminopropyl)-3-ethylcarbodiimide hydrochloride (EDC·HCl, 0.92 g, 4.8 mmol) was added and the solution was stirred at 0° C. for 10 minutes. The mixture was then allowed to react for 12 hours at room temperature in the dark and an N₂ atmosphere. The suspension was subsequently filtered and washed with 1.2 M HCl and saturated NaHCO₃, then dried over anhydrous Na₂SO4. The coarse residue was purified with column chromatography (CH₂Cl₂: EtOAc=4:1). The yellow solid was obtained (0.936 g, 3.17 mmol, yield %=83%). Melting point (mp), 70-73° C. The characterization using NMR Spectroscopy and Mass Spectrometry yielded the following: 1H NMR (CDCl₃, 400 MHZ): δ 7.20 (d, J=9.0 Hz, 1H), 6.50 (dd, J=2.3 Hz and 9.0 Hz, 1H), 6.42 (d, J=2.3 Hz, 1H), 6.00 (s, 1H), 5.14 (s, 2H), 3.33 (q, J=7.0 Hz, 4H), 2.11 (s, 3H), 1.12 (t, J=7.0 Hz, 6H). LCMS (m/z) calculated for C₁₆H₁₉NO₄+ 290.3 found 290.3.

Synthesis of 7-Diethylamino-4-methylacetoate-thiocoumarin 17 was accomplished by the following steps. 7-Diethylamino-4-methylacetoate-coumarin 16 (0.8 g, 2.7 mmol) was dissolved in dry toluene (50 mL) in a flame-dried flask, and Lawesson's reagent (0.74 g, 1.8 mmol) was mixed under N₂. The mixture was refluxed in the dark for 12 hours. The mixture was cooled to room temperature and the solvent was then removed under reduced pressure. The crude residue was purified by column chromatography, using CH₂Cl₂ as the eluent, to afford a dark orange powder (0.647 g, 2.14 mmol, yield %=69%). The characterization using NMR Spectroscopy and Mass Spectrometry yielded the following: 1H NMR (CDCl₃, 400 MHZ): δ 7.31 (d, J=9.7 Hz, 1 H), 7.02 (s, 1H), 6.63 (m, 2H), 5.15 (s, 2H), 3.40 (q, J=7.0 Hz, 4H), 2.16 (s, 3H), 1.19 (t, J=7.0 Hz, 6H). LCMS (m/z) calculated for C₁₆H₂₀NO₃S+ [M+H]⁺ 306.1 found 306.1.

Synthesis of 7-Diethylamino-4-hydroxymethyl-thiocoumarin 18 was accomplished by the following steps. 7-Diethylamino-4-methylacetoate-thiocoumarin 17 (0.5 g, 1.65 mmol) was added to a flame-dried flask and dissolved in absolute ethanol (50 mL). A solution of 1.25 M HCl in ethanol (3.5 mL) was then mixed. The mixture was refluxed in the dark and N₂ atmosphere for 15 hours. The solvent was then removed under reduced pressure and the crude residue was purified by column (CH₂Cl₂: acetone=95:5) to afford a dark brown solid (0.17 g, 0.64 mmol, yield %=30%). The characterization using NMR Spectroscopy and Mass Spectrometry yielded the following: 1H NMR (400 MHZ, acetone-d): δ 7.34 (d, J=8.7 Hz, 1H), 7.27 (s, 1H), 7.06 (s, 1H), 6.68 (s, 1H), 5.19 (s, 2H), 3.42 (t, J=7.1 Hz, 4H), 1.23 (t, J=7.1 Hz, 6H). LCMS (m/z) calculated for C₁₄H₁₈NO₂S⁺ [M+H]⁺ 264.1 found 264.1.

Synthesis of Di-tert-butyl (7-diethylamino-4-carboxylmethyl-thiocoumarin) glutamate 19 was accomplished by the following steps. 7-Diethylamino-4-hydroxymethyl-thiocoumarin 18 and t-butyl isocyanate glutamate 13 (214 mg, 0.75 mmol) were added to a flame-dried flask in toluene (30 mL) under an N₂ atmosphere. After adding the catalytic amount of triethylamine, the whole system was heated to 50° C. and stirred under N₂ for 72 hours. The reaction mixture was then diluted with EtOAc and washed with saturated NH₄Cl and brine. The aqueous layer was extracted with EtOAc and the organic phase was dried with anhydrous Na₂SO₄. After the solvent was removed, the compound was purified by column chromatography (hexane: acetone=4:1). The final product was then obtained by removing the solvent under vacuum (79 mg, 0.14 mmol, yield %=76%). The characterization using NMR Spectroscopy and Mass Spectrometry yielded the following: 1H NMR (400 MHZ, CDCl₃): δ 7.35 (d, J=8.8 Hz, 1H), 7.06 (s, 1H), 6.66 (dt, J=8.7, 2.7 Hz, 2H), 5.56 (d, J=8.1 Hz, 1H), 5.19 (d, J=5.6 Hz, 2H), 4.28 (td, J=8.1, 4.8 Hz, 1H), 3.43 (q, J=7.1 Hz, 4H), 2.47-2.19 (m, 2H), 1.68-1.54 (m, 2H), 1.46 (d, J=12.1, 4.6 Hz, 18H), 1.33-1.09 (t, 6H). LCMS (m/z) calculated for C₂₈H₄₀N₂O₇S⁺ [M+H]⁺ 549.2 found 549.2.

Lastly, synthesis of 7-Diethylamino-4-carboxylmethyl-thiocoumarin glutamate 1 was accomplished by the following steps. Di-tert-butyl (7-diethylamino-4-carboxylmethyl-thiocoumarin) glutamate 19 (30 mg, 0.055 mmol) was added to a flame-dried flask and mixed with trifluoroacetic acid (TFA) (1 mL) at 0° C. The solution was stirred in the dark for 2 hours as it warmed up to room temperature. Upon completion, the TFA was removed with an N₂ stream. The crude residue was purified by reverse-phase HPLC using 60% water, 40% acetonitrile, and 0.1% TFA as mobile phases with an isocratic elution at a rate of 1 mL/min. After collecting the product, the solution was concentrated and then freeze dried by a lyophilizer affording compound 1 (12 mg, 0.028 mmol, yield %=50%). The characterization using NMR Spectroscopy and Mass Spectrometry yielded the following: 1H NMR (400 MHZ, CDCl₃) δ 7.35 (d, J=13.2 Hz, 1H), 7.06 (s, 1H), 6.67 (s, 1H), 6.64 (d, J=2.4 Hz, 1H), 5.56 (d, J=12.7 Hz, 1H), 5.19 (d, J=12.5 Hz, 1H), 4.28 (td, J=8.0, 5.0 Hz, 1H), 3.43 (q, J=7.1 Hz, 4H), 2.43-2.27 (m, 2H), 1.68-1.53 (t, 2H), 1.22 (t, J=7.1 Hz, 6H). ¹³C NMR (101 MHZ, CDCl₃): δ 180.9 (s), 174.8 (s), 163.4 (s), 156.6 (s), 144.6 (s), 141.9 (s), 140.2 (s), 134.4 (s), 123.7 (s), 122.1 (s), 100.9 (s), 69.8 (s), 55.9 (s), 48.4 (s), 34.9 (s), 29.6 (s), 15.6 (s). LCMS (m/z) calculated for C₂₀H₂₄N₂O₇S⁺ [M+H]⁺ 437.1304 found 437.1304.

### General Procedure for Synthesis of Me-OH-BODIPY Derivatives

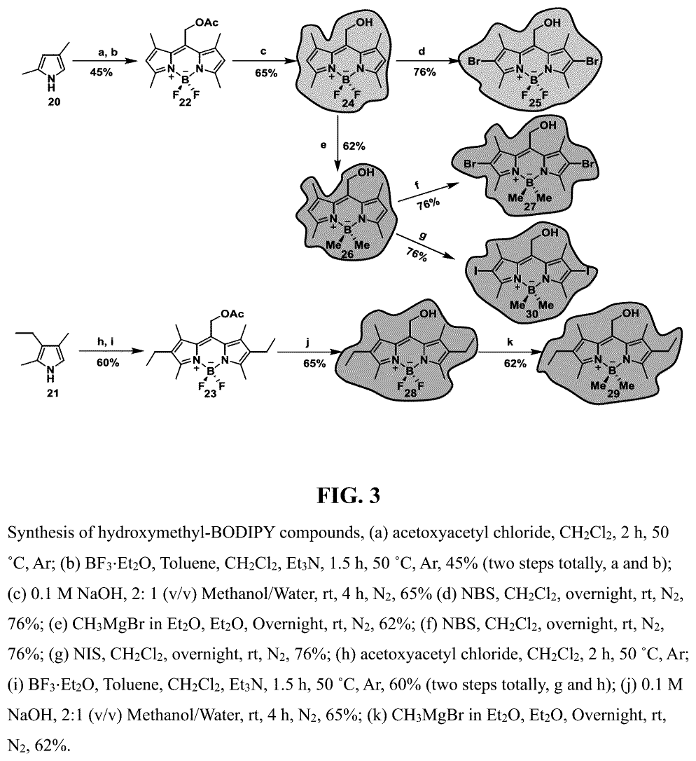

Referring to FIG. 3, shown is a procedure and intermediates for synthesizing hydroxymethyl-BODIPY compounds. Synthesis of 4,4′-Difluoro-8-methylacetoate-1,3,5,7-tetramethyl-4-bora-3a,4a-diaza-s-indacene 22 was accomplished by the following steps. 2,4-Dimethylpyrrole 20 (1.00 g, 10.5 mmol) was added in a flame-dried flask with anhydrous CH₂Cl₂ (50 mL) under Ar, then acetoxyacetyl chloride (0.72 g, 5.26 mmol) was added to the solution and heated at 50° C. in the dark. After 2 hours, the solvent was removed. Toluene (100 mL) and CH₂Cl₂ (10 mL) were added immediately, and the air was displaced with Ar for 30 minutes at room temperature. Triethylamine (2.55 g, 25.0 mmol) was then added dropwise. Finally, BF3 in ether (5.14 g, 36.2 mmol) was added, and the whole system was heated at 50° C. for 1.5 hours under Ar. After the solvent was removed, the residue was purified by column chromatography (silica gel, hexane: EtOAc=4:1), and the final product was obtained by recrystallization from methanol at −20° C. to generate a dark pink solid (0.78 g, 2.4 mmol, yield %=45%). The characterization using NMR Spectroscopy and Mass Spectrometry yielded the following: 1H NMR (400 MHZ, CDCl₃): δ=2.40 (s, 6H), 2.60 (s, 6H), 5.30 (s, 2H), 6.00 (s, 2H). LCMS (m/z) calculated for C₁₆H₁₉BF₂N₂O₂⁺ [M+H]⁺ 321.2 found 321.2.

Synthesis of 2,6-Diethyl-4,4′-difluoro-8-methylacetoate-1,3,5,7-tetramethyl-4-bora-3a, 4a-diaza-s-indacene 23 was accomplished by the following steps. 2,4-dimethyl-3-ethylpyrrole 21 (1.00 g, 8.15 mmol) was added in the flame-dried flask to an anhydrous CH₂Cl₂ (50 mL) under Ar. Then acetoxyacetyl chloride (0.56 g, 4.08 mmol) solution was added dropwise and heated at 50° C. in the dark. After 2 hours, the solvent was removed. Toluene (100 mL) and CH₂Cl₂ (10 mL) were added immediately, and the air was displaced with an Ar stream for 30 minutes at room temperature. Then triethylamine was added dropwise (1.98 g, 1.94 mmol). Finally, BF₃ (3.98 g, 28 mmol) was added to the ether, and the whole system was heated at 50° C. under Ar for 1.5 hours. After the solvent was removed, the residue was purified by column chromatography (silica gel, hexane: EtOAc=4:1), and the final product was obtained by recrystallization at −20° C. with methanol to generate a dark pink solid (0.85 g, 2.2 mmol, yield %=54%). The characterization using NMR Spectroscopy and Mass Spectrometry yielded the following: 1H NMR (400 MHZ, CDCl₃): δ=1.30 (t, J=7.5 Hz, 6H), 2.20 (s, 3H), 2.40 (s, 3H), 2.50 (q, J=7.5 Hz, 4H), 2.60 (s, 6H), 5.30 (s, 2H). LCMS (m/z) calculated for C₂₀H₂₈BF₂N₂O₂⁺ [M+H]⁺ 377.2 found 377.2

Synthesis of 4,4′-Difluoro-8-hydroxymethyl-1,3,5,7-tetramethyl-4-bora-3a,4a-diaza-s-indacene 24 was accomplished by the following steps. 4,4′-Difluoro-8-methylacetoate-1,3,5,7-tetramethyl-4-bora-3a,4a-diaza-s-indacene 22 (500 mg, 1.52 mmol) was added to the round bottom flask with CH₂Cl₂ (20 mL) and methanol (40 mL). Then 0.1 M NaOH (6.50 mmol, 1.3 mL) solution was added dropwise. After stirring under N₂ at room temperature for 4 hours in the dark, the solvent was removed, the crude product was dissolved in CH₂Cl₂, and then washed twice with saturated NH₄Cl and brine respectively. The aqueous layer was then extracted twice with CH₂C₁₂, and the crude was dried using anhydrous Na₂SO4. After column purification (hexane: EtOAc=4:1), the product (273 mg, 0.71 mmol, yield %=65%) was obtained. The characterization using NMR Spectroscopy and Mass Spectrometry yielded the following: 1H NMR (400 MHZ, CDCl₃): δ 6.09 (s, 2H), 4.91 (d, J=5.4 Hz, 2H), 2.52 (d, J=7.6 Hz, 12H). ¹³C NMR (101 MHz, CDCl₃): δ 156.6 (s), 153.4 (s), 144.6 (s), 141.9 (s), 140.2 (s), 134.4 (s), 123.7 (s), 113.1 (s), 100.9 (s), 55.9 (s), 24.4 (s), 16.7 (s), 15.5 (s), 14.6 (s). LCMS (m/z) calculated for C₁₄H₁₈BF₂N₂O⁺ [M+H]⁺ 279.1 found 279.1.

Synthesis of 2,6-Dibromo-4,4′-difluoro-8-hydroxymethyl-1,3,5,7-tetramethyl-4-bora-3a,4a-diaza-s-indacene 25 was accomplished by the following steps. 4,4′-Difluoro-8-hydroxymethyl-1,3,5,7-tetramethyl-4-bora-3a,4a-diaza-s-indacene 24 (50 mg, 0.18 mmol), N-bromosuccinimide (NBS, 80 mg, 0.45 mmol), and CH₂Cl₂ (20 mL) were mixed in a round bottom flask and stirred overnight at room temperature. Upon reaction completion, the mixture was washed with water and then dried over anhydrous Na₂SO₄. The organic layer was removed by vacuum and the crude was purified by column (silica gel, hexane: EtOAc=4:1) to produce a dark pink solid (60 mg, 0.14 mmol, yield %=76%). The characterization using NMR Spectroscopy and Mass Spectrometry yielded the following: 1H NMR (400 MHZ, acetone): δ 4.97 (s, 2H), 2.59 (s, 6H), 2.54 (s, 6H). ¹³C NMR (101 MHz, CDCl₃): δ 163.4 (s), 156.7 (s), 144.6 (s), 142.0 (s), 140.2 (s), 134.4 (s), 122.1 (s), 113.1 (s), 100.9 (s), 55.9 (s), 24.4 (s), 16.7 (s), 15.6 (s), 14.7 (s). LCMS (m/z) calculated for C₁₄H₁₆BBr₂F₂N₂O⁺ [M+H]⁺ 434.9 found 434.9.

Synthesis of 4,4′-Dimethyl-8-hydroxymethyl-1,3,5,7-tetramethyl-4-bora-3a,4a-diaza-s-indacene 26 was accomplished by the following steps. 4,4′-Difluoro-8-hydroxymethyl-1,3,5,7-tetramethyl-4-bora-3a,4a-diaza-s-indacene 24 (100 mg, 0.36 mmol) was added to a flame-dried flask and dissolved by anhydrous ether (50 mL). Then 3 M of methylmagnesium bromide ether solution (1.2 mL, 3.6 mmol) was added dropwise and the mixture was stirred overnight at room temperature in the dark under an N₂ atmosphere. After the reaction was completed as confirmed by TLC, the solution was quenched by adding water. The solution was then washed with saturated NH₄Cl and extracted with CH₂Cl₂. The organic layer was washed with brine and dried with anhydrous Na₂SO₄. The solvent was then removed under vacuum and the residue was purified by column chromatography (hexane: EtOAc=4:1). After removing the solvent, a dark pink solid (60 mg, 0.22 mmol, yield %=62%) is produced. The characterization using NMR Spectroscopy and Mass Spectrometry yielded the following: 1H NMR (400 MHZ, CDCl₃): δ 6.09 (s, 2H), 4.96 (d, J=5.6 Hz, 2H), 2.53 (s, 6H), 2.46 (s, 6H), 0.68-0.31 (s, 6H). ¹³C NMR (101 MHZ, CDCl₃): δ 152.8 (s), 138.3 (s), 136.9 (s), 130.6 (s), 122.5 (s), 56.4 (s), 16.5 (s), 15.9 (s). LCMS (m/z) calculated for C₁₆H₂₃BN₂O⁺ [M+H]⁺ 271.2 found 271.2.

Synthesis of 2,6-Dibromo-4,4′-dimethyl-8-hydroxymethyl-1,3,5,7-tetramethyl-4-bora-3a,4a-diaza-s-indacene 27 was accomplished by the following steps. 4,4′-Dimethyl-8-hydroxymethyl-1,3,5,7-tetramethyl-4-bora-3a,4a-diaza-s-indacene 26 (18 mg, 0.067 mmol), NBS (30 mg, 0.17 mmol), and CH₂Cl₂ (10 mL) were mixed in a round bottom flask and stirred overnight at room temperature. After the reaction is finished, the solution was washed with water and then dried over anhydrous Na₂SO₄. The organic layer was removed in vacuum, and the crude residue was purified by column chromatography (silica gel, hexane: EtOAc=4:1) to generate a dark pink solid (20 mg, 0.047 mmol, yield %=76%). The characterization using NMR Spectroscopy and Mass Spectrometry yielded the following: 1H NMR (400 MHZ, CDCl₃): δ 4.98 (s, 2H), 2.55 (s, 6H), 2.51 (s, 6H), 0.19 (s, 6H). ¹³C NMR (101 MHZ, CDCl₃): δ 150.9 (s), 138.0 (s), 134.4 (s), 129.7 (s), 112.6 (s), 56.7 (s), 56.7 (s), 15.8 (s), 14.9 (s). LCMS (m/z) calculated for C₁₆H₂₂BBr₂N₂O⁺ [M+H]⁺ 427.0 found 427.0.

Synthesis of 2,6-Diethyl-4,4′-difluoro-8-hydroxymethyl-1,3,5,7-tetramethyl-4-bora-3a, 4a-diaza-s-indacene 28 was accomplished by the following steps. 2,6-Diethyl-4,4′-difluoro-8-methylacetoate-1,3,5,7-tetramethyl-4-bora-3a,4a-diaza-s-indacene 23 (500 mg, 1.30 mmol) was added to the round bottom flask with CH₂Cl₂ (20 mL) and methanol (40 mL). Then 0.1 M NaOH (6.50 mmol, 1.3 mL) was added dropwise into the solution. After 4 hours of stirring under N₂ at room temperature in the dark, the solvent was removed. The crude product was dissolved in CH₂Cl₂ and then washed twice with NH₄Cl and brine, respectively. Then the aqueous layer was extracted twice with CH₂Cl₂, and the crude product was dried with anhydrous Na₂SO₄. After column purification (hexane: EtOAc=4:1), the product was obtained (273 mg, 0.82 mmol, yield %=63%). The characterization using NMR Spectroscopy and Mass Spectrometry yielded the following: 1H NMR (400 MHZ, CDCl₃): δ 5.32 (s, 2H), 2.51 (s, 6H), 2.39 (q, J=7.6 Hz, 4H), 2.26 (s, 6H), 1.05 (t, J=7.6 Hz, 6H). ¹³C NMR (101 MHz, CDCl₃): δ 155.3 (s), 153.5 (s), 146.4 (s), 142.6 (s), 138.2 (s), 134.5 (s), 122.2 (s), 111.0 (s), 104.2 (s), 63.6 (s), 29.8 (s), 24.5 (s), 19.1 (s), 16.8 (s), 15.7 (s), 14.8 (s). LCMS (m/z) calculated for C₁₈H₂₆BF2N₂O⁺ [M+H]⁺ 335.2 found 335.2.

Synthesis of 2,6-Diethyl-4,4′-dimethyl-8-hydroxymethyl-1,3,5,7-tetramethyl-4-bora-3a,4a-diaza-s-indacene 29 was accomplished by the following steps. 2,6-Diethyl-4,4′-difluoro-8-hydroxymethyl-1,3,5,7-tetramethyl-4-bora-3a,4a-diaza-s-indacene 28 (200 mg, 0.6 mmol) was added to a flame-dried flask and dissolved in anhydrous ether (100 mL). Then 3 M methylmagnesium bromide in ether (2 mL, 6 mmol) was added dropwise and the mixture was stirred overnight at room temperature in the dark under an N₂ atmosphere. After the reaction was completed and monitored by TLC, it was quenched by dropping water. Then the solution was washed with saturated NH₄Cl and extracted with CH₂Cl₂. The organic layer was washed with brine and dried by anhydrous Na₂SO₄. The solvent was then removed under vacuum after filtration and the residue was purified by column chromatography (hexane: EtOAc=4:1). After the solvent was removed, a dark pink solid (132 mg, 0.4 mmol, yield %=67%) was produced. The characterization using NMR Spectroscopy and Mass Spectrometry yielded the following: 1H NMR (400 MHZ, CDCl₃): δ 4.99 (s, 2H), 2.45 (s, 6H), 2.43 (s, 6H), 1.25 (m, 4H), 1.05 (t, J=7.6 Hz, 6H), 0.19 (s, 6H). ¹³C NMR (101 MHz, CDCl₃): δ 151.3 (s), 136.5 (s), 133.1 (s), 131.8 (s), 130.1 (s), 56.8 (s), 29.7 (s), 17.5 (s), 14.7 (s), 14.5 (s), 12.8 (s). LCMS (m/z) calculated for C₂₀H₃₁BN₂O⁺ [M+H]⁺ 327.3 found 327.3.

Synthesis of 2,6-Diiodo-4,4′-dimethyl-8-hydroxymethyl-1,3,5,7-tetramethyl-4-bora-3a,4a-diaza-s-indacene 30 was accomplished by the following steps. 4,4′-Dimethyl-8-hydroxymethyl-1,3,5,7-tetramethyl-4-bora-3a,4a-diaza-s-indacene 26 (18 mg, 0.067 mmol), N-iodosuccinimide (NIS, 30 mg, 0.17 mmol), and CH₂Cl₂ (10 mL) were mixed in a round bottom flask and stirred overnight at room temperature. After the reaction is finished, the solution was washed with water and then dried over anhydrous Na₂SO₄. The organic layer was removed by vacuum, and the crude residue was purified by column chromatography (silica gel, hexane: EtOAc=4:1) to generate a dark pink solid (20 mg, 0.047 mmol, yield %=76%). The characterization using NMR Spectroscopy and Mass Spectrometry yielded the following: 1H NMR (400 MHZ, CDCl₃): δ 4.98 (d, J=1.8 Hz, 2H), 2.59 (s, 6H), 2.55 (s, 6H), 0.19 (s, 6H). ¹³C NMR (101 MHZ, CDCl₃): δ 153.5 (s), 139.4 (s), 137.2 (s), 130.5 (s), 87.3 (s), 56.9 (s), 18.4 (s), 18.4 (s). LCMS (m/z) calculated for C₁₆H₂₁BI₂N₂O⁺ [M+H]⁺ 523.0 found 523.0.

### General Procedure for Synthesis of BODIPY-Glu Caged Molecules.

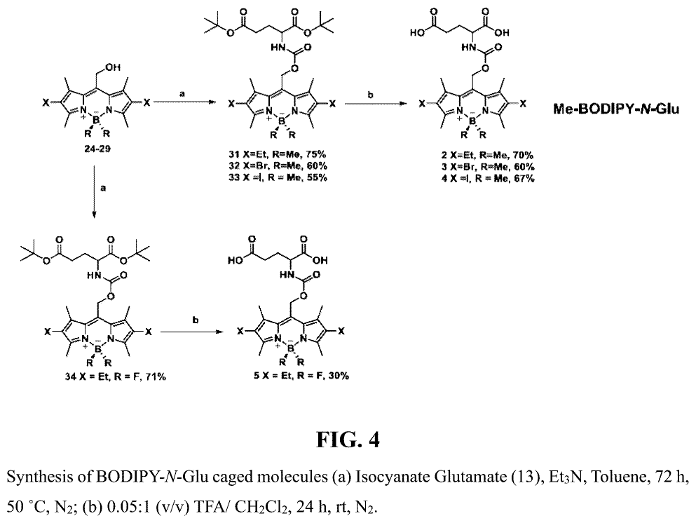

Referring now to FIG. 4, shown is a general synthesis procedure of compounds 31-34. meso-hydroxymethyl-BODIPY 27-30 and t-butylisocyanate glutamate 13 were added to a flame-dried flask with toluene (10 mL) under N₂. After adding the catalytic amount of triethylamine, the reaction mixture was heated to 50° C. and stirred under N₂ for 72 hours. After the reaction is finished, the organic mixture was diluted with EtOAc, and then washed with saturated NH₄Cl followed by brine. The aqueous fraction was extracted with EtOAc, and the organic layer was dried with anhydrous Na₂SO₄. After the solvent was removed, the compound was purified by column chromatography (hexane: acetone=4:1). The solvent was then removed under vacuum to obtain the final product.

The characterization of Di-tert-butyl (((2,8-diethyl-1,3,5,5,7,9-hexamethyl-5H-414,514-dipyrrolo[1,2-c: 2′, 1′-f] [1,3,2] diazaborinin-10-yl) methoxy) carbonyl) glutamate 31 using NMR Spectroscopy and Mass Spectrometry yielded the following: 1H NMR (400 MHZ, CDCl₃) δ 5.41 (t, J=9.1 Hz, 2H), 5.34 (d, J=11.6 Hz, 1H), 4.28 (td, J=8.2, 5.0 Hz, 1H), 2.43 (s, 6H), 2.40 (t, J=7.6 Hz, 2H), 2.31 (s, 6H), 2.13 (m, 2H), 1.89 (m, 2H) 1.71-1.48 (m, 4H), 1.45 (18H), 1.05 (t, J=7.5 Hz, 6H), 0.20 (s, 6H). ¹³C NMR (101 MHz, CDCl₃) δ 177.1 (s), 175.1 (s), 163.9 (s), 159.9 (s), 156.9 (s), 154.1 (s), 149.4 (s), 146.0 (s), 141.1 (s), 132.5 (s), 131.4 (s), 109.1 (s), 88.6 (s), 65.5 (s), 64.1 (s), 33.8 (s), 30.4 (s), 29.5 (s), 22.6 (s), 19.6 (s), 18.9 (s), 16.9 (s), 13.2 (s), 10.9 (s), 9.9 (s). LCMS (m/z) calculated for C₃₄H₅₄BN₃O₆⁺ [M+H]⁺ 612.6 found 612.6.

The characterization of Di-tert-butyl (((2,8-dibromo-1,3,5,5,7,9-hexamethyl-5H-414,514-dipyrrolo[1,2-c: 2′, 1′-f] [1,3,2] diazaborinin-10-yl) methoxy) carbonyl) glutamate 32 using NMR Spectroscopy and Mass Spectrometry yielded the following: 1H NMR (400 MHZ, CDCl₃) δ 5.51 (d, J=7.9 Hz, 1H), 5.36 (d, J=4.7 Hz, 2H), 4.39 (dd, J=13.4, 8.2 Hz, 1H), 4.17 (m, 4H), 2.47 (18H), 1.28 (m, 12H), 0.20 (s, 6H). ¹³C NMR (101 MHZ, CDCl₃) δ 172.5 (s), 172.5 (s), 171.5 (s), 155.7 (s), 151.7 (s), 133.3 (s), 133.3 (s), 132.0 (s), 132.0 (s), 131.5 (s), 130.7 (s), 130.7 (s), 97.1 (s), 61.7 (s), 60.7 (s), 30.2 (s), 30.1 (s), 27.6 (s), 17.5 (s), 14.7 (s), 14.5 (s), 14.1 (s), 12.7 (s). LCMS (m/z) calculated for C₃₀H₄₄BBr₂N₃O₆⁺ [M+H]⁺ 714.3 found 714.3.

The characterization of Di-tert-butyl (((2,8-diiodo-1,3,5,5,7,9-hexamethyl-5H-414,514-dipyrrolo[1,2-c: 2′, 1′-f] [1,3,2] diazaborinin-10-yl) methoxy) carbonyl) glutamate 33 using NMR Spectroscopy and Mass Spectrometry yielded the following: 1H NMR (400 MHZ, CDCl₃)δ 5.52 (dd, J=2.6 Hz, 1H), 5.37 (d, J=5.3 Hz, 1H), 5.13 (s, 2H), 4.22 (dd, J=7.1 Hz, 2H), 4.14 (dd, J=7.2 Hz, 2H), 2.50 (18H), 1.29 (m, 12H), 0.20 (s, 6H). ¹³C NMR (101 MHZ, CDCl₃)δ 172.6 (s), 171.5 (s), 155.5 (s), 155.5 (s), 155.4 (s), 151.7 (s), 139.3 (s), 137.6 (s), 132.9 (s), 131.9 (s), 130.4 (s), 123.9 (s), 85.9 (s), 61.8 (s), 60.8 (s), 30.2 (s), 29.7 (s), 27.5 (s), 18.2 (s), 18.1 (s), 16.7 (s), 16.2 (s), 14.2 (s). LCMS (m/z) calculated for C₃₀H₄₄BI₂N₃O₆⁺ [M+H]⁺ 808.3 found 808.3.

The characterization of Di-tert-butyl (((2,8-diethyl-5,5-difluoro-1,3,7,9-tetramethyl-5H-414,514-dipyrrolo[1,2-c: 2′, 1′-f] [1,3,2] diazaborinin-10-yl) methoxy) carbonyl) glutamate 34 using NMR Spectroscopy and Mass Spectrometry yielded the following: 1H NMR (400 MHZ, CDCl₃) δ 5.34 (d, J=2.7 Hz, 2H), 4.45 (d, J=15.7 Hz, 1H), 3.64 (dd, J=5.8, 3.5 Hz, 1H), 2.50 (s, 6H), 2.37 (dd, J=16.4, 8.1 Hz, 4H), 2.29 (s, 6H), 1.25 (s, 18H), 1.03 (dd, J=14.5, 7.4 Hz, 4H), 0.88 (t, J=7.9 Hz, 6H). ¹³C NMR (101 MHZ, CDCl₃) δ 179.0 (s), 172.6 (s), 160.9 (s), 155.5 (s), 151.3 (s), 149.2 (s), 139.2 (s), 132.8 (s), 127.2 (s), 122.3 (s), 110.9 (s), 107.6 (s), 82.3 (s), 72.0 (s), 63.5 (s), 35.9 (s), 31.9 (s), 29.9 (s), 22.6 (s), 20.7 (s), 18.8 (s), 15.9 (s), 14.7 (s), 10.6 (s). LCMS (m/z) calculated for C₃₂H₄₈BF₂N₃O₆⁺ [M+H]⁺ 620.6 found 620.6.

Referring to FIGS. 1 & 4, the general synthesis procedure of Me-BODIPY-N-Glu 2-4 and F-BODIPY-N-Glu 5 was accomplished by the following steps. Compounds 31-34 were added to a flame-dried flask and mixed with anhydrous CH₂Cl₂ (1 mL). The solution was stirred in a 0° C. ice bath for 10 minutes. Then TFA (50 μL) was added dropwise, and the solution was stirred under N₂ in the dark for 24 h and allowed to warm up to room temperature. After the reaction was finished, the solvent was removed under vacuum. The crude residues were purified by reverse phase HPLC using 20% water, 80% acetonitrile, and 0.1% TFA as the mobile phase with an isocratic elution rate of 1 mL/min. After collecting the products, the solution was concentrated and the aqueous layer was dried with a lyophilizer to generate the final products: Me-BODIPY-Et-N-Glu 2, Me-BODIPY—Br-N-Glu 3, F-BODIPY—I-N-Glu 4, and F-BODIPY-Et-N-Glu 5.

The characterization of (((2,8-Diethyl-1,3,5,5,7,9-hexamethyl-5H-414,514-dipyrrolo[1,2-c: 2′, 1′-f] [1,3,2] diazaborinin-10-yl) methoxy) carbonyl) glutamic acid 2 using NMR Spectroscopy and Mass Spectrometry yielded the following: 1H NMR (400 MHZ, CDCl₃) δ 5.43 (t, J=9.1 Hz, 2H), 4.29 (td, J=8.2, 5.0 Hz, 1H), 2.44 (s, 6H), 2.43-2.37 (m, 2H), 2.33 (s, 6H), 2.14 (m, 2H), 1.90 (m, 2H), 1.76-1.50 (m, 4H), 1.06 (t, J=7.5 Hz, 6H), 0.22 (s, 6H). ¹³C NMR (101 MHz, CDCl₃) δ 177.1 (s), 175.2 (s), 164.0 (s), 159.9 (s), 156.9 (s), 154.1 (s), 149.4 (s), 146.1 (s), 141.2 (s), 132.5 (s), 131.5 (s), 109.1 (s), 65.5 (s), 64.1 (s), 33.9 (s), 30.4 (s), 22.7 (s), 19.6 (s), 18.9 (s), 16.9 (s), 13.2 (s), 10.9 (s), 10.0 (s). HRMS (m/z) calculated for C₂₆H₃₈BN₃O₆⁺ [M+H]⁺ 500.4151 found 500.4151.

The characterization of (((2,8-Dibromo-1,3,5,5,7,9-hexamethyl-5H-414,514-dipyrrolo[1,2-c: 2′, 1′-f] [1,3,2] diazaborinin-10-yl) methoxy) carbonyl) glutamic acid 3 using NMR Spectroscopy and Mass Spectrometry yielded the following: 1H NMR (400 MHZ, CDCl₃) δ 5.37 (d, J=4.7 Hz, 2H), 4.40 (dd, J=13.4, 8.2 Hz, 1H), 4.18 (ddd, J=23.1, 13.9, 6.9 Hz, 4H), 1.29 (dd, J=17.2, 7.0 Hz, 12H), 0.21 (s, 6H). ¹³C NMR (101 MHZ, CDCl₃) δ 172.5 (s), 172.5 (s), 171.5 (s), 155.7 (s), 151.7 (s), 133.3 (s), 133.3 (s), 132.0 (s), 132.0 (s), 131.5 (s), 130.8 (s), 130.8 (s), 61.7 (s), 60.7 (s), 30.2 (s), 30.2 (s), 17.5 (s), 14.7 (s), 14.5 (s), 14.2 (s), 12.7 (s). HRMS (m/z) calculated for C₂₂H₂₈BBr₂N₃O₆⁺ [M+H]⁺ 602.0990 found 602.0990.

The characterization of (((2,8-Diiodo-1,3,5,5,7,9-hexamethyl-5H-414,514-dipyrrolo[1,2-c: 2′, 1′-f] [1,3,2] diazaborinin-10-yl) methoxy) carbonyl) glutamic acid 4 using NMR Spectroscopy and Mass Spectrometry yielded the following: 1H NMR (400 MHZ, CDCl₃) δ 5.53 (dd, J=2.6 Hz, 1H), 5.37 (d, J=5.3 Hz, 2H), 5.14 (s, 2H), 4.23 (dd, J=7.1 Hz, 2H), 4.14 (dd, J=7.2 Hz, 2H), 1.29 (dd, J=16.9, 7.3 Hz, 12H), 0.21 (s, 6H). ¹³C NMR (101 MHZ, CDCl₃) δ 172.5 (s), 171.4 (s), 155.5 (s), 155.4 (s), 155.4 (s), 151.7 (s), 139.2 (s), 137.5 (s), 132.9 (s), 131.9 (s), 130.3 (s), 123.9 (s), 61.7 (s), 60.8 (s), 30.2 (s), 29.7 (s), 18.2 (s), 18.0 (s), 16.7 (s), 16.2 (s), 14.2 (s). HRMS (m/z) calculated for C₂₂H₂₈BI₂N₃O₆⁺ [M+H]⁺ 696.0999 found 696.0999.

The characterization of (((2,8-Diethyl-5,5-difluoro-1,3,7,9-tetramethyl-5H-414,514-dipyrrolo[1,2-c: 2′, 1′-f] [1,3,2] diazaborinin-10-yl) methoxy) carbonyl) glutamic acid 5 using NMR Spectroscopy and Mass Spectrometry yielded the following: 1H NMR (400 MHZ, CDCl₃) δ 5.44 (d, J=2.7 Hz, 2H), 3.74 (dd, J=5.8, 3.5 Hz, 1H), 2.47 (dd, J=16.4, 8.1 Hz, 4H), 2.39 (s, 6H), 2.13 (s, 6H), 1.74 (m, 4H), 0.98 (t, J=6.7 Hz, 6H). ¹³C NMR (101 MHZ, CDCl₃) δ 179.1 (s), 172.8 (s), 160.9 (s), 155.6 (s), 151.4 (s), 149.3 (s), 139.4 (s), 132.9 (s), 127.3 (s), 122.4 (s), 111.1 (s), 107.8 (s), 72.1 (s), 63.6 (s), 36.0 (s), 32.0 (s), 22.7 (s), 20.8 (s), 18.9 (s), 16.0 (s), 14.8 (s), 10.7 (s). HRMS (m/z) calculated for C₂₄H₃₂BF₂N₃O₆⁺ [M+H]⁺ 508.3418. found 508.3418.

### General Synthesis Procedure of Compounds 35-40.

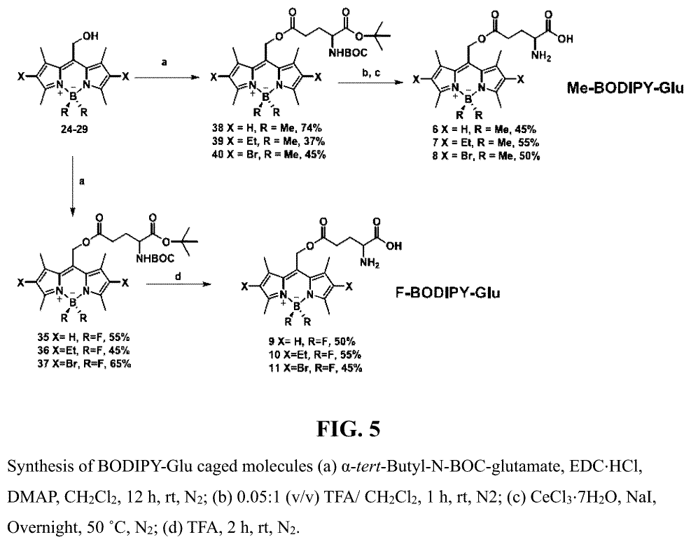

Referring to FIG. 5, meso-Hydroxymethyl-BODIPY 24-29 were added to a flame-dried flask and dissolved in anhydrous CH₂Cl₂. Then a-tert-butyl-N-BOC-glutamate and 4-dimethylaminopyridine (DMAP) were added and stirred under N₂. The ice bath was employed to cool down the solution to 0° C. Thereafter, 1-(3-dimethylaminopropyl)-3-ethylcarbodiimide HCl (EDC· HCl) was added, and the mixture was stirred at 0° C. for 10 minutes. The mixture was then allowed to react for 12 hours at room temperature in the dark and under N₂ atmosphere. After the reaction was finished and monitored by TLC, the suspension was filtered. The organic filtrate was washed with 1.2 M HCl and saturated with NaHCO₃, then dried over anhydrous Na₂SO₄. The crude residue was purified by column chromatography (CH₂Cl₂: EtOAc=4:1).

The characterization of 1-(tert-Butyl) 5-((5,5-difluoro-1,3,7,9-tetramethyl-5H-414,514-dipyrrolo[1,2-c: 2′, 1′-f] [1,3,2] diazaborinin-10-yl)methyl) (tert-butoxycarbonyl) glutamate 35 using NMR Spectroscopy and Mass Spectrometry yielded the following: 1H NMR (400 MHZ, CDCl₃) δ 6.08 (s, 2H), 5.37-5.28 (q, 2H), 5.07 (d, J=8.2 Hz, 1H), 4.22 (dd, J=12.3, 7.4 Hz, 1H), 2.53 (s, 6H), 2.45 (m, 2H), 2.36 (s, 6H), 2.03 (m, 2H), 1.44 (d, J=12.5 Hz, 18H). ¹³C NMR (101 MHZ, CDCl₃) δ 176.1 (s), 173.4 (s), 162.7 (s), 153.5 (s), 146.2 (s), 139.4 (s), 137.2 (s), 130.5 (s), 121.1 (s), 104.4 (s), 102.2 (s), 100.6 (s), 87.3 (s), 61.0 (s), 56.7 (s), 30.9 (s), 29.7 (s), 27.1 (s), 19.8 (s), 18.4 (s), 18.4 (s), 15.3 (s). LCMS (m/z) calculated for C₂₈H₄₀BF₂N₃O₆⁺ [M+H]⁺ 564.4 found 564.4.

The characterization of 1-(tert-Butyl) 5-((2,8-diethyl-5,5-difluoro-1,3,7,9-tetramethyl-5H-414,514-dipyrrolo[1,2-c: 2′, 1′-f] [1,3,2] diazaborinin-10-yl)methyl) (tert-butoxycarbonyl) glutamate 36 using NMR Spectroscopy and Mass Spectrometry yielded the following: 1H NMR (400 MHZ, CDCl₃) δ 5.34 (s, 2H), 5.08 (d, J=7.6 Hz, 1H), 4.22 (t, J=5.9 Hz, 1H), 2.51 (s, 6H), 2.39 (dd, J=15.1, 7.5 Hz, 4H), 2.25 (s, 6H), 1.46 (s, 9H), 1.43 (s, 9H), 1.30 (q, J=21.6, 13.2 Hz, 4H), 1.05 (t, J=7.5 Hz, 6H). ¹³C NMR (101 MHZ, CDCl₃) δ 174.9 (s), 170.8 (s), 165.1 (s), 155.5 (s), 153.2 (s), 151.4 (s), 136.6 (s), 133.2 (s), 131.9 (s), 130.3 (s), 121.8 (s), 105.5 (s), 84.9 (s), 56.9 (s), 50.9 (s), 29.9 (s), 28.8 (s), 26.9 (s), 19.7 (s), 17.6 (s), 14.9 (s), 14.5 (s), 12.9 (s), 9.8 (s), 8.6 (s). LCMS (m/z) calculated for C₃₂H₄₈BF₂N₃O₆⁺ [M+H]⁺ 620.6 found 620.6.

The characterization of 1-(tert-Butyl) 5-((2,8-dibromo-5,5-difluoro-1,3,7,9-tetramethyl-5H-414,514-dipyrrolo[1,2-c: 2′, 1′-f] [1,3,2] diazaborinin-10-yl)methyl) (tert-butoxycarbonyl) glutamate 37 using NMR Spectroscopy and Mass Spectrometry yielded the following: 1H NMR (400 MHZ, CDCl₃) δ 5.34 (s, 2H), 5.07 (dd, J=8.8, 1.1 Hz, 1H), 4.23 (d, J=1.9 Hz, 1H), 2.59 (s, 6H), 2.55-2.41 (m, 2H), 2.39 (s, 6H), 2.21 (m, 2H), 1.46 (s, 9H), 1.42 (s, 9H). ¹³C NMR (101 MHz, CDCl₃) δ 178.8 (s), 173.7 (s), 168.1 (s), 159.4 (s), 146.7 (s), 139.3 (s), 136.8 (s), 133.7 (s), 131.8 (s), 122.6 (s), 118.1 (s), 103.3 (s), 87.8 (s), 57.3 (s), 54.4 (s), 34.1 (s), 29.3 (s), 24.1 (s), 18.1 (s), 15.6 (s), 11.2 (s), 8.4 (s). LCMS (m/z) calculated for C₂₈H₃₈BBr₂F₂N₃O₆⁺ [M+H]⁺ 722.2 found 722.2.

The characterization of 1-(tert-Butyl) 5-((1,3,5,5,7,9-hexamethyl-5H-414,514-dipyrrolo[1,2-c: 2′, 1′-f] [1,3,2] diazaborinin-10-yl)methyl) (tert-butoxycarbonyl) glutamate 38 using NMR Spectroscopy and Mass Spectrometry yielded the following: 1H NMR (400 MHZ, CDCl₃) δ 6.08 (s, 2H), 5.36 (d, J=2.4 Hz, 2H), 5.06 (dd, J=8.4, 1.9 Hz, 1H), 4.96 (d, J=5.3 Hz, 1H), 2.46 (s, 6H), 2.35 (s, 6H), 2.26-2.16 (dd, 2H), 1.97 (m, 2H), 1.45 (s, 9H), 1.42 (s, 9H), 0.19 (s, 6H). ¹³C NMR (101 MHZ, CDCl₃) δ 173.8 (s), 172.9 (s), 152.9 (s), 141.4 (s), 138.3 (s), 136.9 (s), 130.6 (s), 128.3 (s), 122.5 (s), 115.6 (s), 111.9 (s), 102.2 (s), 89.2 (s), 62.7 (s), 56.4 (s), 32.4 (s), 29.7 (s), 26.7 (s), 23.9 (s), 19.8 (s), 17.8 (s), 16.5 (s), 15.9 (s). LCMS (m/z) calculated for C₃₀H₄₆BN₃O₆⁺ [M+H]⁺ 556.6 found 556.6.

The characterization of 1-(tert-Butyl) 5-((2,8-diethyl-1,3,5,5,7,9-hexamethyl-5H-414,514-dipyrrolo[1,2-c: 2′, 1′-f] [1,3,2] diazaborinin-10-yl)methyl) (tert-butoxycarbonyl) glutamate 39 using NMR Spectroscopy and Mass Spectrometry yielded the following: 1H NMR (400 MHZ, CDCl₃) δ 5.38 (d, J=1.9 Hz, 2H), 5.08 (d, J=7.9 Hz, 1H), 4.22 (dd, J=11.3, 6.7 Hz, 1H), 2.43 (s, 6H), 2.25 (s, 6H), 2.07-1.70 (m, 4H), 1.45 (s, 9H), 1.43 (s, 9H), 1.38-1.10 (m, 4H), 1.04 (t, J=7.5 Hz, 6H), 0.20 (s, 6H). ¹³C NMR (101 MHz, CDCl₃) δ 172.5 (s), 171.5 (s), 155.7 (s), 151.7 (s), 133.3 (s), 133.3 (s), 132.0 (s), 132.0 (s), 131.5 (s), 131.5 (s), 130.8 (s), 130.7 (s), 97.1 (s), 61.7 (s), 60.7 (s), 30.2 (s), 30.1 (s), 27.6 (s), 17.5 (s), 14.7 (s), 14.5 (s), 14.4 (s), 14.1 (s), 14.1 (s), 12.7 (s). LCMS (m/z) calculated for C₃₄H₅₄BN₃O₆⁺ [M+H]⁺ 612.6 found 612.6.

The characterization of 1-(tert-Butyl) 5-((2,8-dibromo-1,3,5,5,7,9-hexamethyl-5H-414,514-dipyrrolo[1,2-c: 2′, 1′-f] [1,3,2] diazaborinin-10-yl)methyl) (tert-butoxycarbonyl) glutamate 40 using NMR Spectroscopy and Mass Spectrometry yielded the following: 1H NMR (400 MHZ, CDCl₃) δ 5.37 (s, 2H), 5.07 (d, J=8.3 Hz, 1H), 4.23 (dd, J=10.6, 6.6 Hz, 1H), 2.51 (s, 6H), 2.44 (m, 2H), 2.37 (s, 6H), 2.21 (m, 2H), 1.44 (d, J=12.3 Hz, 18H), 0.20 (s, 6H). ¹³C NMR (101 MHZ, CDCl₃) δ 173.5 (s), 170.9 (s), 167.4 (s), 150.9 (s), 141.2 (s), 139.0 (s), 138.0 (s), 134.4 (s), 131.7 (s), 130.4 (s), 129.7 (s), 112.6 (s), 83.5 (s), 68.0 (s), 56.7 (s), 35.5 (s), 32.7 (s), 29.7 (s), 18.9 (s), 15.8 (s), 14.9 (s), 13.5 (s), 9.8 (s). LCMS (m/z) calculated for C₃₀H₄₄BBr₂N₃O₆⁺ [M+H]⁺ 714.3 found 714.3.

### General Synthesis Procedure Me-BODIPY-Glu 6-8.

Referring to FIGS. 1 & 5, compounds 38-40 were added to a flame-dried flask and mixed with anhydrous CH₂Cl₂ (1 mL). The solution was stirred in an ice bath (0° C.) for 10 min. Then TFA (50 μL) was added dropwise, and the solution was stirred under N₂ in the dark for 1 hour while allowing it to warm up to room temperature. Upon completion, the solvent was removed. The suspension was then refluxed with CeCl₃·7H₂O (1.5 mmol) and NaI (1.3 mmol) in acetonitrile (30 mL) for 24 hours. After reflux, the solution was cooled to room temperature, 1.5 mL of suspension was added to a 2-dram vial and stirred overnight at 95° C. Then 0.5 N HCl was added to quench the reaction and the solvent was subsequently removed under vacuum. The crude residue was purified by reverse phase HPLC using 20% water, 80% acetonitrile, and 0.1% TFA with an isocratic elution rate of 1 mL/min. After the product was collected, the solution was concentrated and the aqueous layer was dried with lyophilizer to generate the final products: Me-BODIPY-H-Glu 6, Me-BODIPY-Et-Glu 7, Me-BODIPY-Br-Glu 8.

The characterization of 2-Amino-5-((1,3,5,5,7,9-hexamethyl-5H-414,514-dipyrrolo[1,2-c: 2′, 1′-f] [1,3,2] diazaborinin-10-yl) methoxy)-5-oxopentanoic acid 6 using NMR Spectroscopy and Mass Spectrometry yielded the following: 1H NMR (400 MHZ, CDCl₃) δ 6.09 (s, 2H), 5.36 (d, J=2.4 Hz, 2H), 5.07 (dd, J=8.4, 1.9 Hz, 1H), 2.47 (s, 6H), 2.35 (s, 6H), 2.19 (m, 2H), 1.98 (m, 2H), 0.20 (s, 6H). ¹³C NMR (101 MHZ, CDCl₃) δ 173.9 (s), 173.0 (s), 152.9 (s), 141.6 (s), 138.4 (s), 137.1 (s), 130.7 (s), 128.4 (s), 122.6 (s), 115.7 (s), 112.0 (s), 102.4 (s), 62.8 (s), 56.5 (s), 32.5 (s), 29.8 (s), 24.1 (s), 19.9 (s), 17.9 (s), 16.7 (s), 16.0 (s). HRMS (m/z) calculated for C₂₁H₃₀BN₃O₄⁺ [M+H]⁺ 400.29801 found 400.29801.

The characterization of 2-Amino-5-((2,8-diethyl-1,3,5,5,7,9-hexamethyl-5H-414,514-dipyrrolo[1,2-c: 2′, 1′-f] [1,3,2] diazaborinin-10-yl) methoxy)-5-oxopentanoic acid 7 using NMR Spectroscopy and Mass Spectrometry yielded the following: 1H NMR (400 MHZ, CDCl₃) δ 5.38 (d, J=1.9 Hz, 2H), 4.22 (dd, J=12.4, 7.8 Hz, 1H), 2.43 (s, 6H), 2.40 (dd, J=12.4, 4.9 Hz, 4H), 2.25 (s, 6H), 1.91 (m, 4H), 1.04 (t, J=7.5 Hz, 6H), 0.20 (s, 6H). ¹³C NMR (101 MHZ, CDCl₃) δ 172.5 (s), 171.5 (s), 155.7 (s), 151.7 (s), 133.3 (s), 133.3 (s), 132.0 (s), 132.0 (s), 131.5 (s), 131.5 (s), 130.8 (s), 130.7 (s), 61.7 (s), 60.7 (s), 30.2 (s), 30.2 (s), 17.5 (s), 14.7 (s), 14.5 (s), 14.4 (s), 14.2 (s), 14.1 (s), 12.7 (s). HRMS (m/z) calculated for C₂₅H₃₈BN₃O₄⁺ [M+H]⁺ 456.4060 found 456.4060.

The characterization of 2-Amino-5-((2,8-dibromo-1,3,5,5,7,9-hexamethyl-5H-414,514-dipyrrolo[1,2-c: 2′, 1′-f] [1,3,2] diazaborinin-10-yl) methoxy)-5-oxopentanoic acid 8 using NMR Spectroscopy and Mass Spectrometry yielded the following: 1H NMR (400 MHZ, CDCl₃) δ 5.38 (s, 2H), 4.24 (dd, J=10.6, 6.6 Hz, 1H), 2.52 (s, 6H), 2.38 (s, 6H), 2.22 (dd, J=11.9, 5.6 Hz, 2H), 2.06-1.76 (m, 2H), 0.21 (s, 6H). ¹³C NMR (101 MHZ, CDCl₃) δ 173.6 (s), 171.0 (s), 167.5 (s), 151.1 (s), 141.3 (s), 139.1 (s), 138.1 (s), 134.5 (s), 131.8 (s), 130.5 (s), 129.8 (s), 112.7 (s), 68.1 (s), 56.8 (s), 35.6 (s), 32.8 (s), 18.9 (s), 15.9 (s), 15.1 (s), 13.6 (s), 9.9 (s). HRMS (m/z) calculated for C₂₁H₂₈BBr₂N₃O₄⁺ [M+H]⁺ 558.09001 found 558.09001.

### General Synthesis Procedure of F-BODIPY-Glu 9-11.

Referring to FIGS. 1 & 5, compounds 35-37 were added to a flame-dried flask and mixed with TFA (1 mL) at 0° C. The solution was stirred under N₂ in the dark for 2 hours while allowing it to warm up to room temperature. After the reaction was finished, the TFA was removed with an N₂ stream. Crude residues were purified by reverse phase HPLC using 20% water, 80% acetonitrile, and 0.1% TFA as mobile phases with an isocratic elution rate of 1 mL/min. After the product was collected, the solution was concentrated and then dried with a lyophilizer to generate the final products: F-BODIPY-H-Glu 9, F-BODIPY-Et-Glu 10, F-BODIPY-Br-Glu 11.

The characterization of 2-Amino-5-((5,5-difluoro-1,3,7,9-tetramethyl-5H-414,514-dipyrrolo[1,2-c: 2′, 1′-f] [1,3,2] diazaborinin-10-yl) methoxy)-5-oxopentanoic acid 9 using NMR Spectroscopy and Mass Spectrometry yielded the following: 1H NMR (400 MHZ, CDCl₃) δ 6.08 (s, 2H), 5.41-5.19 (m, 2H), 4.22 (dd, J=12.3, 7.4 Hz, 1H), 2.53 (s, 6H), 2.45 (m, 2H), 2.36 (s, 6H), 2.18 (m, 1H), 1.88 (m, 1H). ¹³C NMR (101 MHZ, CDCl₃) δ 176.2 (s), 173.5 (s), 162.8 (s), 153.6 (s), 146.3 (s), 139.5 (s), 137.3 (s), 130.6 (s), 121.2 (s), 104.6 (s), 102.3 (s), 100.8 (s), 61.1 (s), 57.1 (s), 31.0 (s), 29.8 (s), 19.9 (s), 18.5 (s), 18.5 (s), 15.4 (s). HRMS (m/z) calculated for C₁₉H₂₄BF₂N₃O₄⁺ [M+H]⁺ 408.2248 found 408.2248.

The characterization of 2-Amino-5-((2,8-diethyl-5,5-difluoro-1,3,7,9-tetramethyl-5H-414,514-dipyrrolo[1,2-c: 2′, 1′-f] [1,3,2] diazaborinin-10-yl) methoxy)-5-oxopentanoic acid 10 using NMR Spectroscopy and Mass Spectrometry yielded the following: 1H NMR (400 MHZ, CDCl₃) δ 5.44 (s, 2H), 4.32 (t, J=5.9 Hz, 1H), 2.61 (s, 6H), 2.49 (m, 4H), 2.35 (s, 6H), 1.41 (m, 4H), 1.15 (t, J=7.5 Hz, 6H). ¹³C NMR (101 MHZ, CDCl₃) δ 174.9 (s), 170.8 (s), 165.1 (s), 155.5 (s), 153.1 (s), 151.4 (s), 136.6 (s), 133.2 (s), 131.9 (s), 130.2 (s), 121.8 (s), 105.5 (s), 56.9 (s), 50.9 (s), 29.8 (s), 28.8 (s), 19.6 (s), 17.6 (s), 14.8 (s), 14.6 (s), 12.9 (s), 9.8 (s), 8.6 (s). HRMS (m/z) calculated for C₂₃H₃₂BF₂N₃O₄[M+H]⁺ 464.3328 found 464.3328.

The characterization of 2-Amino-5-((2,8-dibromo-5,5-difluoro-1,3,7,9-tetramethyl-5H-414,514-dipyrrolo[1,2-c: 2′, 1′-f] [1,3,2] diazaborinin-10-yl) methoxy)-5-oxopentanoic acid 11 using NMR Spectroscopy and Mass Spectrometry yielded the following: 1H NMR (400 MHZ, CDCl₃) δ 5.44 (s, 2H), 5.17 (dd, J=5.4 Hz, 2H), 2.69 (s, 6H), 2.58 (m, 2H), 2.49 (s, 6H), 2.30 (m, 2H). ¹³C NMR (101 MHZ, CDCl₃) δ 178.9 (s), 173.8 (s), 168.2 (s), 159.6 (s), 146.8 (s), 139.4 (s), 136.9 (s), 133.8 (s), 131.9 (s), 122.7 (s), 118.2 (s), 103.4 (s), 57.4 (s), 54.5 (s), 34.2 (s), 29.4 (s), 18.2 (s), 15.7 (s), 11.3 (s), 8.5 (s). HRMS (m/z) calculated for C₁₉H₂₂BBr₂F₂N₃O₄⁺ [M+H]⁺ 566.0168 found 566.0168.

### Synthesis of Hydroxymethyl-BODIPY

Still referring to FIG. 3, the BODIPY cage was synthesized using previously reported steps starting from 2,4-dimethylpyrrole 20 or 3-ethyl-2,4-dimethylpyrrole 21. Under the action of acetoxyacetyl chloride followed by BF₃ etherate, the BODIPY frame can be assembled in relatively good yield ranging from about 30-40%.

Still referring to FIG. 3, the inventors initially employed the 2,6-diethylated BODIPY resulting from 21 but noted that replacing the ethyl groups with halogen atoms (Br and I) greatly improved the quantum yield. To achieve substitutions at the 2,6 positions (also referred to as B-positions), compound 24 formed from 20 was used as the general synthon. Treatment of 24 with NBS or NIS resulted in the formation of the brominated 25 and iodinated 26 BODIPY, respectively. The analogs that contained methylated boron were generated by first reacting the key intermediate 24 with CH₃MgBr in ether prior to bromination and iodination at the B-positions, to generate 27 and 30, respectively.

### Synthesis of BODIPY-Cage

The caging procedures of Glu to the varying BODIPY cages involved the common hydroxymethyl intermediate at the meso position (C-8). In general, the Glu was attached via the amino functionality (FIG. 4) or the y-carboxylic acid group (FIG. 5). For N-protection, the diBoc Glu isocyanate was used under triethylamine conditions with mild heat (50° C.) (FIG. 4).

Referring to FIG. 5, for the carboxy attachment, a classic EDC coupling was carried out between the hydroxy methylated BODIPY and the a-tert-butyl-N-BOC Glu.

Still referring to FIG. 5, traditional TFA deprotection typically involves exposing the molecule to TFA at low temperatures and shorter reaction times. TFA is a strong acid that is often used for deprotection of t-butyl protecting groups. Challenges were encountered when the Me-BODIPY system that was attached to the γ-carboxy end of Glu was to be deprotected (intermediates 38-40). In the latter cases, several attempts at standard TFA deprotection resulted in either no reaction (at low temperatures and shorter reaction times) or decomposition when higher temperature and reaction times were employed (see Table S1 below). This is likely due to the presence of sensitive functional groups or multiple protecting groups in these intermediates. We were able to circumvent these challenges in the methylated BODIPY precursors (38-40) by resorting to a 5% TFA in CH₂Cl₂ for 1 hour at room temperature followed by treatment with CeCl₃ and NaI at 50° C. overnight.

TABLE S1
Results of different method employed to deprotect
Me-BODIPY-Glu compounds (38-40)

| System | Temperature/° C. | Time/h | Result |
|---|---|---|---|
| TFA | rt | 1 | Degradation |
| | 0 | 1 | Degradation |
| | −10 | 2 | No Reaction |
| 1:1 (v/v) TFA/DCM | rt | 2 | Degradation |
| | 0 | 2 | Degradation |
| 0.05:1 (v/v) | rt | | Degradation |
| TFA/DCM | 0 | 8 | Degradation |
| K₂CO₃/CH₃OH | 50 | Overnight | No Reaction |
| | Reflux | 4 | Degradation |

### UV Analysis of Caged Molecules

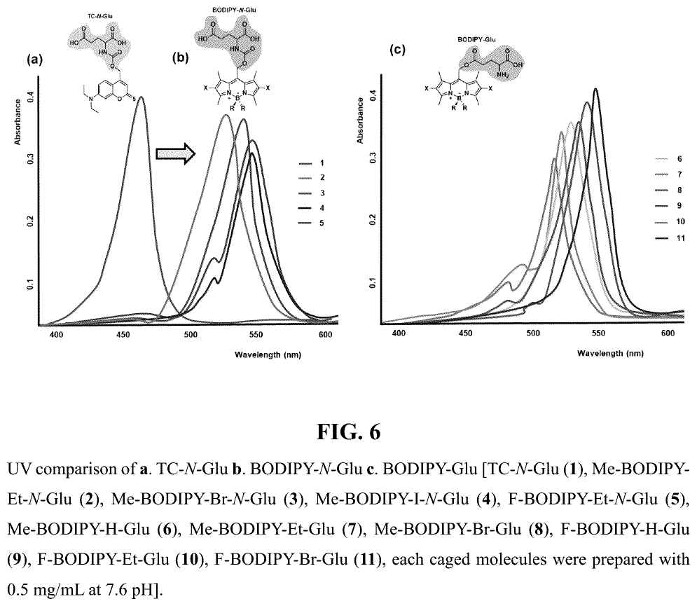

Referring to FIG. 6, a comprehensive assessment of 11 visible light-activated caged Glutamate (Glu) compounds was conducted using photophysical methods. Notably, the thiocoumarin caged Glu, TC-Glu 1, was activated by blue light, at 445 nm, whereas the BODIPY cages displayed distinct relative bathochromic shifts. This led efforts to focus on generating more variants of caged Glu based on the BODIPY system. Cages attached to the amino group of Glu exhibited Amax values spanning 515-540 nm, while those attached to the γ-carboxylic acid group displayed Amax ranges of 520-560 nm.

The inventors studied the kinetics of uncaging of the two caged Glu designs (TC and BODIPY) and compared their efficiency of uncaging under identical conditions. They were irradiated with blue light at 467 nm and green light at 520 nm, respectively. Referring to FIG. 10, a temperature-controlled rig with purchased LED strips was set up.

The process for conducting the irradiation experiments comprised the following:

Sample Preparation: The caged molecules (containing the BODIPY photocage and Glutamate) were prepared in a solvent at a predetermined concentration. The solution was carefully prepared to ensure uniform dispersion of the molecules.

Irradiation Setup: The irradiation experiments were performed in a custom-built irradiation chamber equipped with the necessary safety measures (FIG. 10). The chamber was designed to prevent any unwanted exposure to the irradiation source and to maintain a controlled environment throughout the experiment.

Irradiation Source: The irradiation source used was from LED light Strips. This source was selected based on its wavelength range, intensity, and compatibility with the caged molecules' photochemistry. The source was positioned within the irradiation chamber to ensure an even and consistent illumination of the sample.

Sample Exposure: The sample solution was placed in a suitable container, ensuring that the solution thickness was uniform to minimize potential variations in the irradiation process. The container was positioned at a specific distance from the irradiation source, and the entire setup was shielded from external light sources to prevent interference.

Irradiation Protocol: The sample was irradiated for a predetermined duration, with the irradiation source emitting light at the desired wavelength range. The irradiation time was carefully controlled to maintain consistency across experiments.

Monitoring and Data Collection: Throughout the irradiation process, the changes in the sample's properties were monitored using spectroscopic techniques such as UV-Vis spectroscopy and NMR spectroscopy, depending on the specific properties being investigated. Data was collected at regular intervals to track the progress of the photochemical reaction.

Control Experiments: To validate the effects of irradiation, control experiments were conducted using identical conditions but without the presence of the caged molecules. These control experiments served as a baseline for comparing the observed changes in the sample.

The flux of the LED light strips that were used was calculated to be 12.8 Wm-2. The quantification of the laser flux comprised the following:

A liquid-phase potassium ferrioxalate actinometer was prepared to measure the intensity of light in our irradiation rig, i.e., light flux, necessary for measurement of the quantum yields of various photochemical reactions. The actinometer was synthesized by dissolving 6.0 g of ammonium ferrous sulfate hexahydrate in a beaker containing 20 ml of water and 1 mL of 3 M sulfuric acid. Separately, 3.5 g of oxalic acid dihydrate was mixed with 35 mL of water in another beaker and heated to dissolve. Next, 20 mL of the oxalic acid solution was added dropwise to the ammonium ferrous sulfate solution, which was then heated to 100° C. After cooling, the precipitate was filtered, and it was mixed with 15 mL of saturated potassium oxalate. While heating around 40° C., 12 mL of 6% hydrogen peroxide was added dropwise to the mixture with continuous stirring. After the reaction, the solution was heated to boiling to remove excess hydrogen peroxide, and the compound from step 2 was added until the solution turned green and lucent. Following cooling, 15 mL of 95% ethanol was added for crystallization in the dark, and the solution was subsequently filtered. The resulting solid was recrystallized three times in water, yielding K3Fe(C₂O₄)₃·3H₂O.

To prepare a 100 mL 0.15 M K₃Fe(C₂O₄)₃·3H₂O solution, 7.36 g of the purified crystals were dissolved in 80 mL water, and 10 mL of 1.0 N sulfuric acid were added before transferring it to a 100 mL volumetric flask. The flask was then filled with water up to the mark. All quantitative work, including the preparation of solutions and samples, was conducted in the dark to avoid any side photochemical reactions. The light intensity was determined by irradiating the potassium ferrioxalate solution and monitoring the subsequent changes in absorbance at 510 nm using blue and green LED strips with wavelengths of 467 nm and 520 nm, respectively, set up in the cylindrical metal containers. For each radiation measurement, 3 mL of 0.15 M potassium ferrioxalate solution was filled into a cuvette, which was then placed on the sample rack, stirred, and irradiated for specific time intervals (0, 0.25, 0.50, 0.75, 1.0, 2.0, 3.0 minutes). After each irradiation, the solution was transferred to a 25 mL volumetric flask and mixed with 6 mL of developer solution (0.05 mol % phenanthroline/0.75 M acetate/0.2 M sulfuric acid) and 5 mL of 1 M sodium fluoride aqueous solution. Water was added to reach the 25 mL mark, and the mixture was incubated for 10 minutes. Subsequently, a 3 mL sample was taken into a 1 cm cuvette, and the absorbance at 510 nm was measured using a UV-Vis spectrophotometer at the end of the incubation period.

The flux of the laser was determined using the following equations:

$$I = \frac{Δ n}{1 {0}^{- 3} \cdot Φ \cdot {V}_{1} \cdot t} \qquad ( Equation 1 )$$

Where I represents the flux in Einstein per liter per second (Einstein/L/s), An represents the moles of Fe²⁺ photogenerated, Φ is the known quantum yield at 532 nm, V₁ is the irradiated volume in milliliters (mL), and t is the irradiation time in seconds.

$$Δ n = \frac{1 {0}^{- 3} \cdot {V}_{1} \cdot {V}_{3} \cdot {C}_{T}}{{V}_{2}} \qquad ( Equation 2 )$$

Where V₂ is the volume taken from the irradiated sample in mL, V₃ is the volume after dilution for concentration determination in mL, and CT is the concentration of Fe²⁺ after dilution.

$${C}_{T} = \frac{Abs}{ε \cdot l} \qquad ( Equation 3 )$$

Where Abs is the absorbance at 510 nm, ε is the molar absorptivity (i.e., molar attenuation constant) in M⁻¹ cm⁻¹, and/is the path length.

Equations 1-3 enable the quantification of the laser flux and are essential for determining efficiency and kinetics. Therefore, “Einstein per liter” (Einstein/L) represents the number of moles of photons per liter of solution. The “per second” (1/s) part indicates that the flux measures the number of photons released or absorbed per unit time.

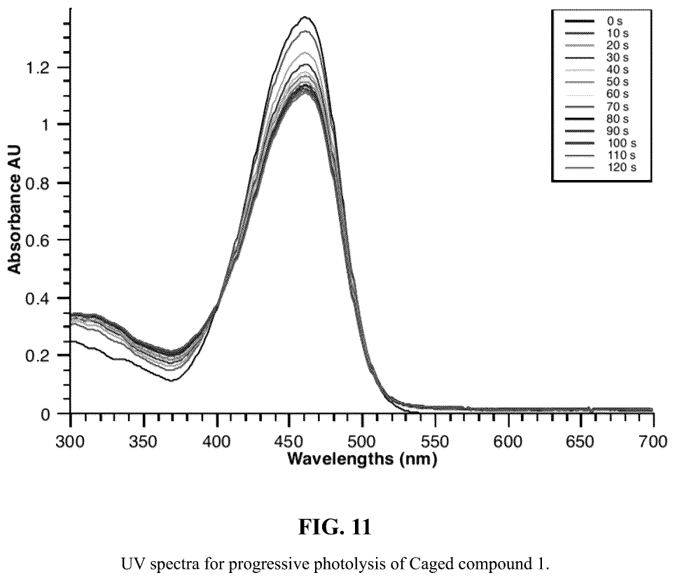

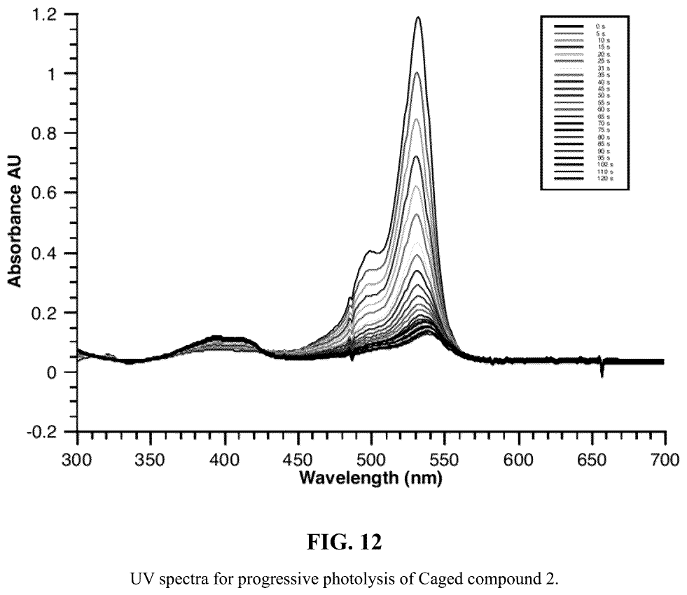

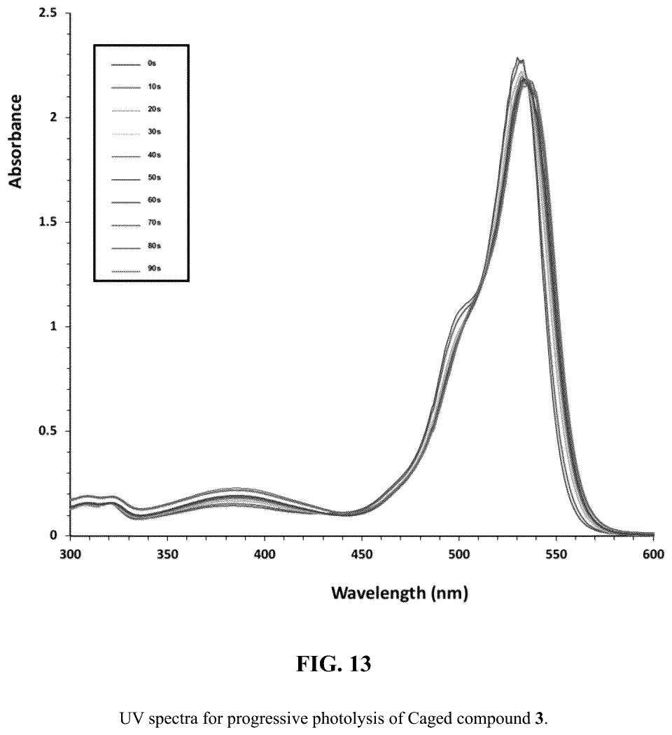

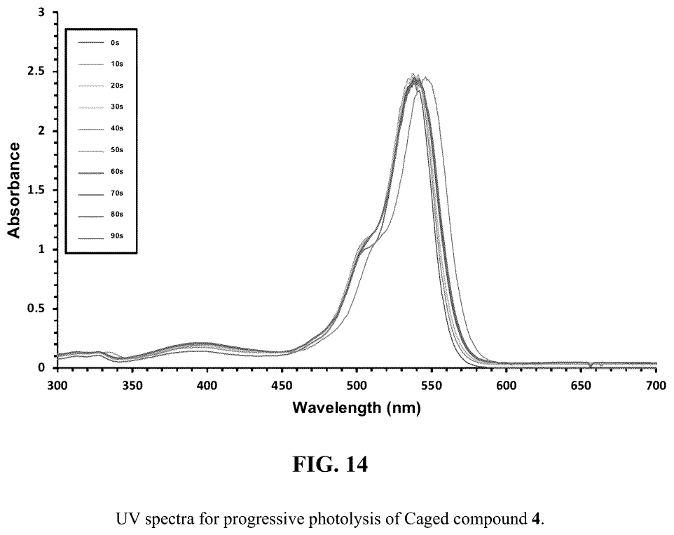

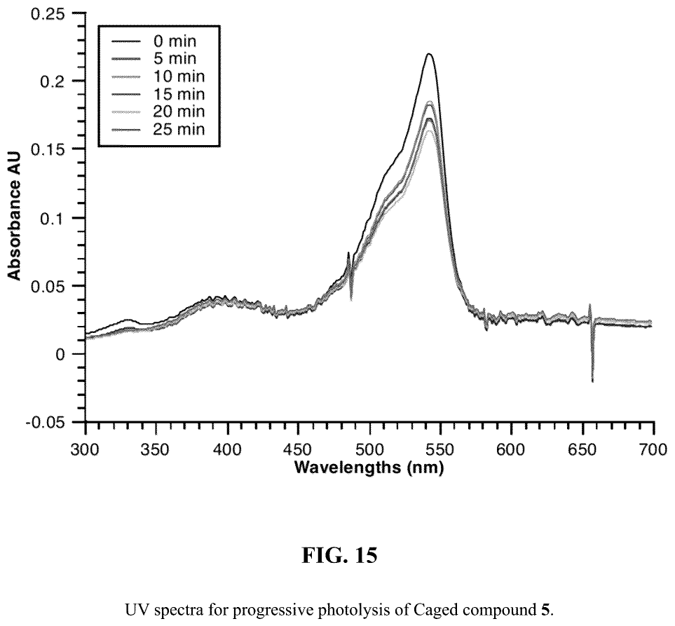

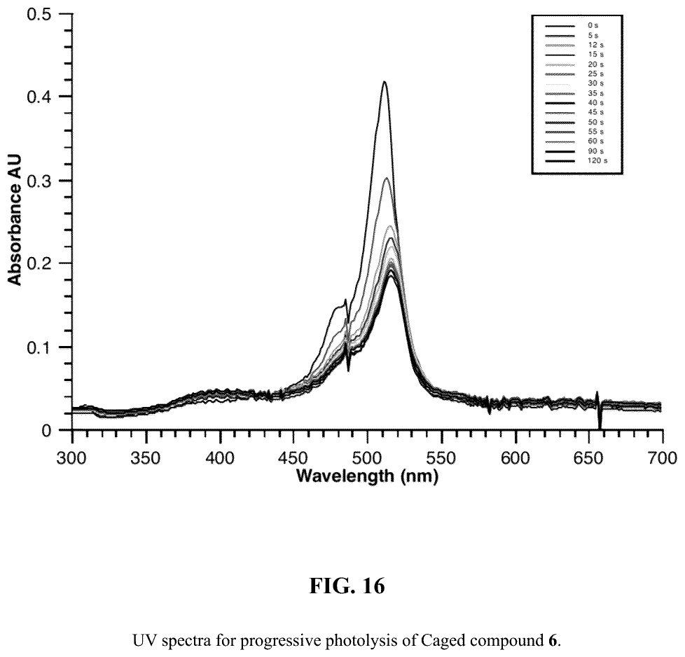

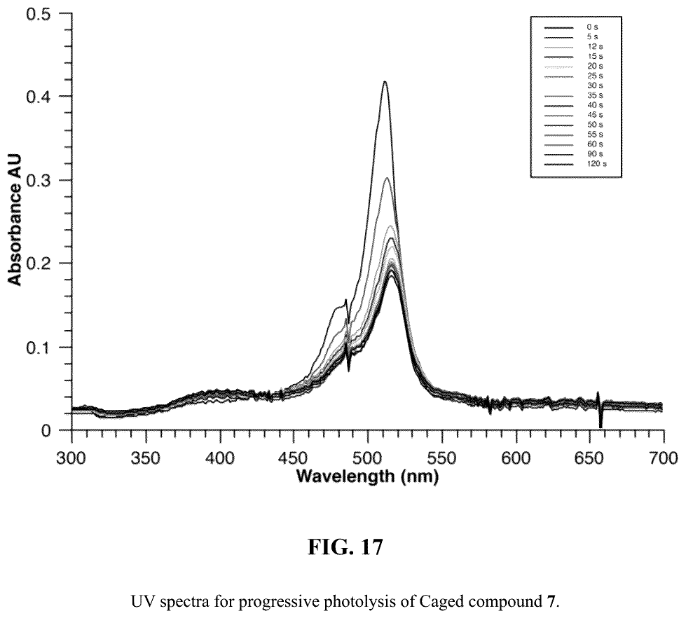

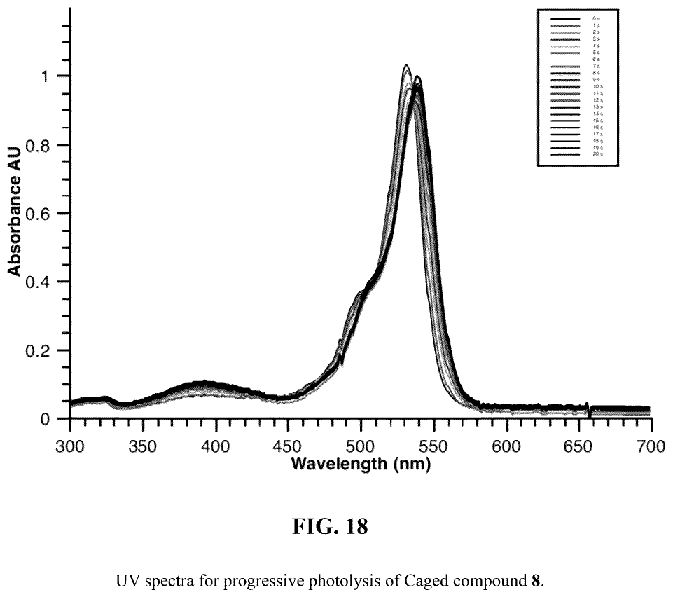

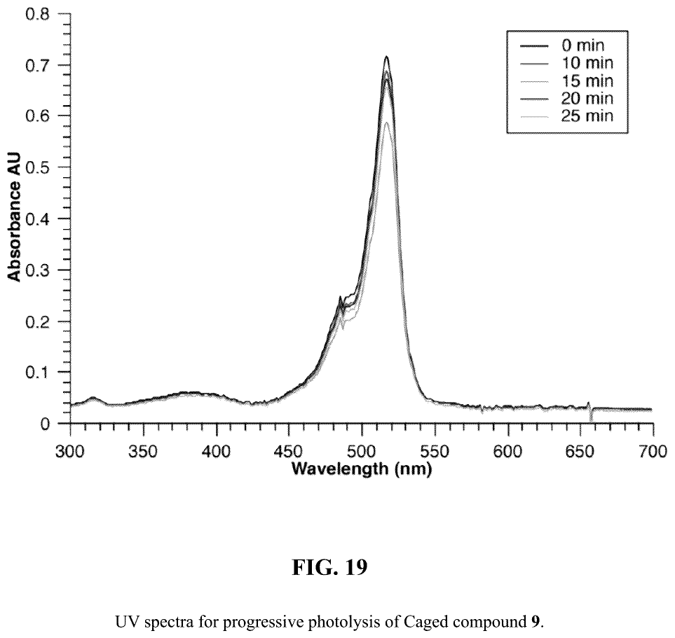

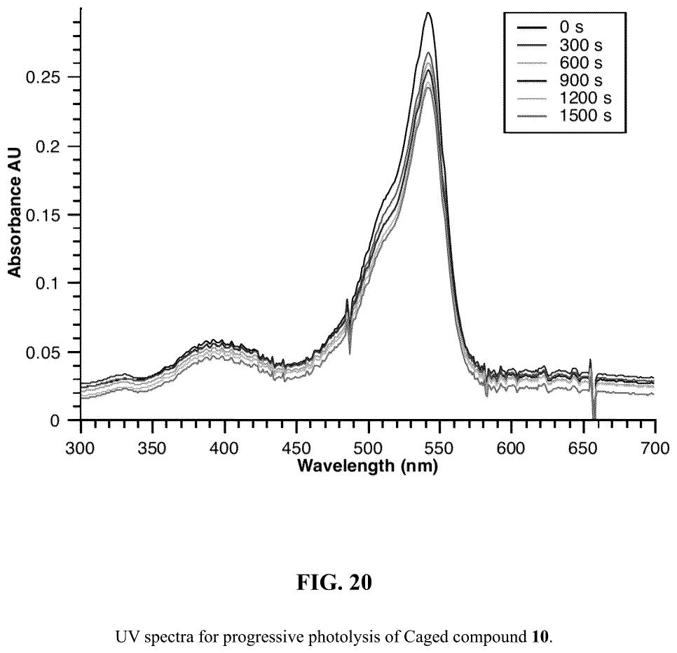

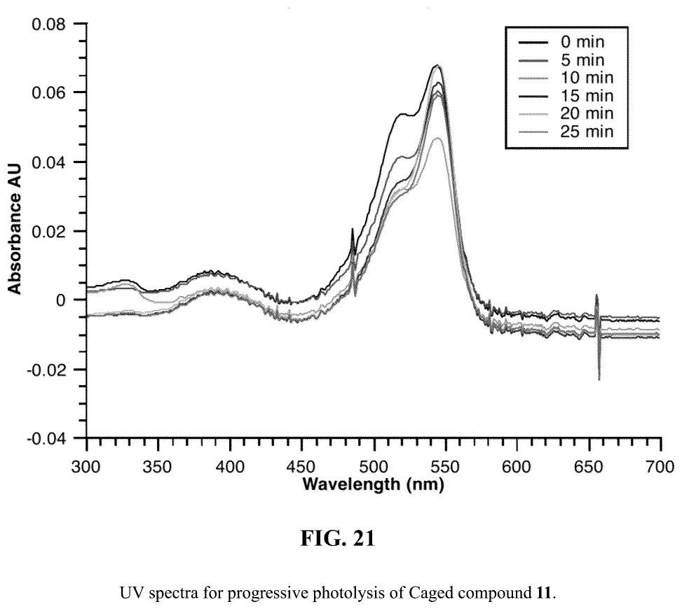

Referring to FIGS. 11-21, irradiation took place in the photo reactor at 23° C. at specified intervals of 10 seconds for progressive UV analysis. It was necessary to irradiate the latter (F-BODIPY-Et-N-Glu 5, F-BODIPY-H-Glu 9, F-BODIPY-Et-Glu 10, F-BODIPY-Br-Glu 11) for a longer time, due to the lower relative quantum yield of some of the BODIPY caged Glu. The absorbance changes, resulting from irradiation experiments, reflect a peak decrease at 430-450 nm, at 525-550 nm, and 500-560 nm for TC-N-Glu, BODIPY-N-Glu and BODIPY-Glu, respectively.

### Quantum Yield Comparison of Caged Molecules

The rate constant for the reactions (k), maximum absorbance wavelength (λₘₐₓ), quantum yields (Φ) and photo-cross-sections (εΦ) were calculated and are presented in Table 1. The results determined that TC-N-Glu has a higher quantum yield and photo-cross-section than F-BODIPY-Et-N-Glu (5), F-BODIPY-H-Glu (9) and F-BODIPY-Br-Glu (11). Efforts were made to address the latter's lower quantum yields (Φ), which included a modified synthesis of a methylated version of that caged, in which we replaced the fluorine atoms on the boron of classic BODIPY with methyl groups. The photophysical studies of this modified caged noted a much-improved quantum yield. The results are summarized in Table 1 below.

Photochemical Properties of Caged Glu by 1-11

| | λ_{Max}/ | ε(λ_{Max})/ | | εΦ/ |
|---|---|---|---|---|
| Caged Glu | nm | M⁻¹cm⁻¹ | Φ | M⁻¹cm⁻¹ |
| TC-N-Glu (1) | 461 | 21800 ± 100 | 0.0017 ± 0.0002 | 38.2 |
| Me-BODIPY- | 532 | 24000 ± 120 | 0.095 ± 0.003 | 2270.0 |
| Et-N-Glu (2) | | | | |
| Me-BODIPY- | 532 | 8213 ± 50 | 0.12 ± 0.04 | 985.6 |
| Br-N-Glu (3) | | | | |
| Me-BODIPY- | 536 | 10030 ± 70 | 0.15 ± 0.01 | 1504.6 |
| I-N-Glu (4) | | | | |
| F-BODIPY- | 542 | 78300 ± 400 | 0.0001 ± 0.00001 | 10.9 |
| Et-N-Glu (5) | | | | |
| Me-BODIPY- | 511 | 43600 ± 200 | 0.074 ± 0.002 | 3230.0 |
| H-Glu (6) | | | | |
| Me-BODIPY- | 532 | 35700 ± 180 | 0.029 ± 0.001 | 1040.0 |
| Et-Glu (7) | | | | |
| Me-BODIPY- | 531 | 54200 ± 240 | 0.65 ± 0.01 | 35230.0 |
| Br-Glu (8) | | | | |
| F-BODIPY-H-Glu (9) | 517 | 84600 ± 500 | 0.0001 ± 0.00002 | 4.7 |
| F-BODIPY- | 542 | 57200 ± 280 | 0.0077 ± 0.0004 | 439.0 |
| Et-Glu (10) | | | | |
| F-BODIPY- | 544 | 10300 ± 80 | 0.0004 ± 0.00001 | 4.2 |
| Br-Glu (11) | | | | |

### Progressive Photolysis of Caged Molecules

Relative photolysis rates of each molecule were analyzed using ¹H NMR for each photochemical irradiation using method previously reported in R.L. Comitz, Y.P. Ouedraogo, N. Nesnas, Unambiguous evaluation of the relative photolysis rates of nitro indolinyl protecting groups critical for brain network studies, Anal. Chem. Res. 3 (2015) 20-25, doi.org/10.1016/j.ancr.2014.11.001 and C. Guruge, S. Y. Rfaish, C. Byrd, S. Yang, A.K. Starrett, E. Guisbert, N. Nesnas, Caged proline in photoinitiated organocatalysis, J. Org. Chem. 84 (2019) 5236-5244, doi.org/10.1021/acs.joc.9b00220; both of which are herein incorporated by reference in their entirety. Compounds were dissolved in the NMR solvent, CDCl₃, at concentrations of 5 mg/mL. The solution was then transferred into a transparent NMR tube and placed into the photo reactor. The TC-N-Glu was irradiated at 467 nm for 60 s intervals and all the 10 variants of BODIPY-(N)-Glu were irradiated at 520 nm for 120 s intervals.

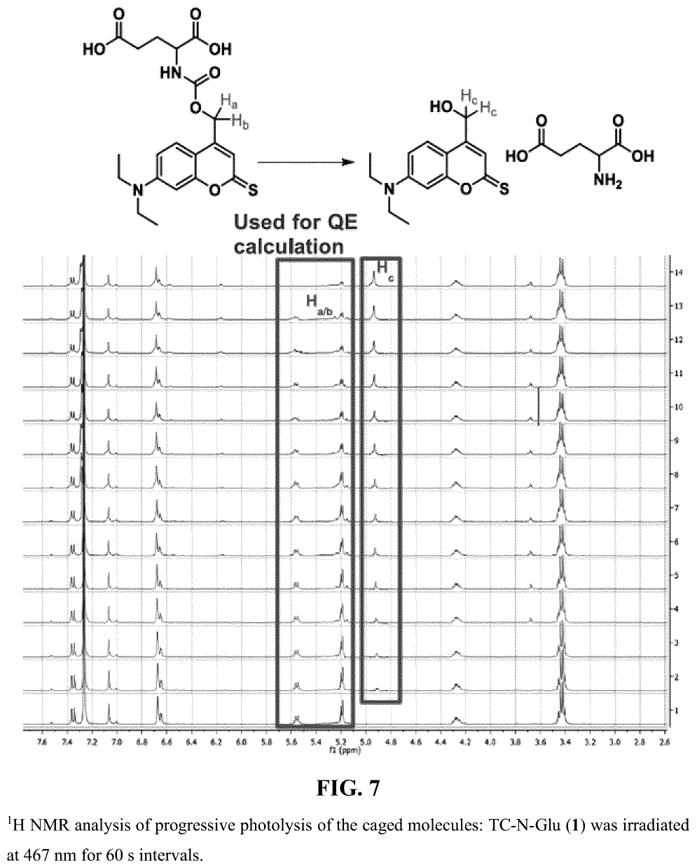

The integration of selected non-exchangeable proton peaks in the caged Glu were used to reflect the concentration of the starting material. Specific quantities of tetramethylsilane (TMS) were applied as internal standard for quantification of caged Glu peaks. Referring to FIGS. 7 & 8, a representative example of the photoirradiation of TC-N-Glu (1) and Me-BODIPY-H-Glu (6) caged Glu. In progressive photo illumination of caged 1, we noticed the Ha/b peaks at 5.2-5.6 ppm decreased with serial irradiation relative to the internal standard. This is due to the cleavage of the carbamate bond during the uncaging process that re-leases glutamate. The methylene protons (labeled Hₐ and H_{b} in FIG. 7) were used in the quantum yield calculations. The enantiotopic protons of the methylene group of TC become diastereotopic due to the nearby chiral center of the Glu. Therefore, they have two different chemical shifts and they also split one another (as shown). Upon irradiation, cleavage of the carbamate releases the Glu and forms the TC with enantiotopic methylene protons that have identical chemical shifts and appear as a singlet labeled He at 4.9 ppm.

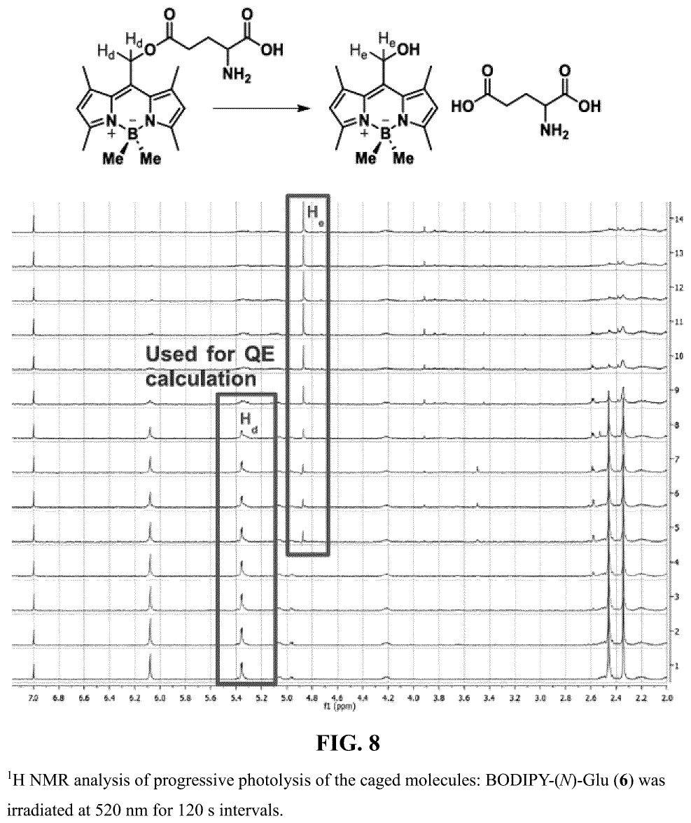

In the photolysis of the BODIPY caged Glu, the diastereotopic methylene protons at 5.36 ppm (labeled H_{d} in FIG. 8) appear as a singlet due to the negligible effect of the remote chiral center of Glu. Upon progressive irradiation, the integration of the H_{d} protons decreases, while the integration of the detached BODIPY methylene protons, Hₑ at 4.9 ppm, appears and increases. The decrease of the integration of H_{d} relative to the internal standard was used in the computations of the QE for all caged Glu compounds.

### CONCLUSION

In this disclosure, the preparation of 11 caged Glu molecules that uncage with visible light is disclosed. One cage design was based on thiocoumarin (TC) and responds to blue light at 467 nm. The other 10 caged Glu molecules respond to green light of wavelengths ranging from 520-560 nm. The Glu was caged with BODIPY either at its amino group (N) or its γ-carboxy functional group. With CDNI-Glu, the caged tethered to the carboxy group was more photo-responsive than the cages attached to the N. There were also two substitution variations on the BODIPY cages: 1. The original fluorinated BODIPY (F-BODIPY); and 2. The modified methylated BODIPY (Me-BODIPY). The Me-BODIPY overall had higher quantum yields (i.e., quantum efficiencies or “QE”). Other variations took advantage of the beta position of BODIPY in which the proton/ethyl was substituted by a heavy atom such as Br or I. Of note is heavy atom moiety combined on the beta site of BODIPY contributed to a redshift (bathochromic shift). For instance, the halogen atom, Br, displayed the most noted redshift whereas the replacement of the fluorine on the boron site with methyl groups resulted in a blueshift (hypsochromic shift) in the absorption wavelength toward UV. Additionally, heavy atoms dramatically improved QE. The highest QE of 0.65 was observed using the brominated Me-BODIPY attached to the γ-carboxy end of Glu (Me-BODIPY-Br-Glu, 8). The latter caged Glu possessing the highest QE that efficiently uncaged Glu at 540 nm was not previously reported.

### Applications

The following comprises non-limiting examples of potential practical uses for the invention:

Neuroscience Research: The caged Glutamate (Glu) compounds responsive to visible light have immediate applications in neuroscience research. They enable researchers to precisely control the release of Glu in neural systems, facilitating the study of synaptic transmission, neuronal signaling, and other aspects of brain function.

Optogenetics: These caged compounds are highly relevant to the field of optogenetics, where light-sensitive molecules are used to manipulate and study neural activity. The ability to uncage Glu using visible light offers a valuable tool for optogenetic experiments aimed at understanding brain circuits and behavior.

Pharmacology and Drug Development: The controlled release of Glu can be utilized for in vitro pharmacological studies. It enables researchers to assess the effects of potential drugs or compounds on glutamatergic signaling, contributing to drug development for neurological disorders.

Biomedical Applications: Caged Glu compounds responsive to visible light may find applications in therapeutic interventions for neurological conditions. The precise and minimally invasive release of Glu could be harnessed for targeted treatments.

Neurological Disease Modeling: These caged compounds can be used to create in vitro models of neurological diseases, aiding in the understanding of conditions like epilepsy, Alzheimer's disease, and Parkinson's disease.

Teaching and Education: The technology could be valuable for educational purposes, allowing students to visualize and understand neural processes and neurotransmission.

Clinical Therapies: While not an immediate application, in the future, caged Glu compounds responsive to visible light might be refined for clinical therapies. Controlled Glu release could be utilized for treatments of neurological disorders, potentially offering a more precise and targeted approach to brain-related conditions.

Precision Medicine: As our understanding of neurobiology and the molecular mechanisms of neurological diseases advances, these caged compounds could play a role in personalized or precision medicine, tailoring treatments to individual patients based on their specific neural profiles.

Expanded Optogenetic Applications: Advances in optogenetics may lead to broader applications for caged Glu compounds. They might be used to explore and manipulate neural circuits in novel ways, potentially aiding in the treatment of mood disorders, addiction, and more.

Neuroprosthetics: In the realm of brain-computer interfaces and neuroprosthetics, these compounds could be employed to enhance neural control over prosthetic devices, restoring mobility and function to individuals with neurological injuries or conditions.

Neurological Monitoring: Future iterations of these compounds could be designed for real-time monitoring of neural activity, potentially revolutionizing brain-machine interfaces and diagnostic tools for neurological diseases.

What is claimed is:

1. A caged glutamate composition, comprising the formula:

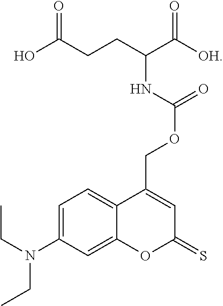 [SMILES: CCN(CC)c1ccc2c(COC(=O)NC(CCC(=O)O)C(=O)O)cc(=S)oc2c1]

2. A caged glutamate composition, comprising the formula: 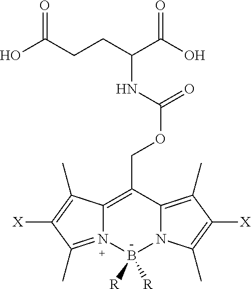
wherein:
X is Bromine, Iodine, or an Ethyl group; and
R is Fluorine or Methyl group.

3. The caged glutamate composition of claim 2, wherein:
X is Bromine; and
R is a Methyl group.

4. The caged glutamate composition of claim 2, wherein:
X is Iodine; and
R is a Methyl group.

5. The caged glutamate composition of claim 2, wherein:
X is Ethyl group; and
R is a Methyl group.

6. The caged glutamate composition of claim 2, wherein:
X is an Ethyl group; and
R is Fluorine.

7. A caged glutamate composition, comprising the formula: 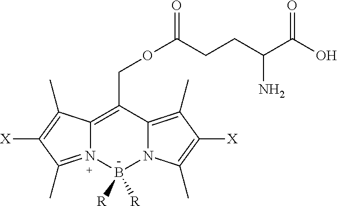
wherein:
X is Hydrogen, Bromine, or an Ethyl group; and
R is Fluorine or a Methyl group.

8. The caged glutamate composition of claim 7, wherein:
X is Hydrogen; and
R is a Methyl group.

9. The caged glutamate composition of claim 7, wherein:
X is Ethyl group; and
R is a Methyl group.

10. The caged glutamate composition of claim 7, wherein:
X is Bromine; and
R is a Methyl group.

11. The caged glutamate composition of claim 7, wherein:
X is Hydrogen; and
R is Fluorine.

12. The caged glutamate composition of claim 7, wherein:
X is an Ethyl group; and
R is Fluorine.

13. The caged glutamate composition of claim 7, wherein:
X is Bromine; and
R is Fluorine.
